SEQUENCE LISTING (19 sequences)

SEQ ID NO: 1 - DNA, 21 bp, Artificial Sequence
  Synthetic polynucleotide
cguuaacauucccuaauuctt

SEQ ID NO: 2 - DNA, 21 bp, Artificial Sequence
  Synthetic polynucleotide
gaauuagggaauguuaacgtg

SEQ ID NO: 3 - DNA, 18 bp, Artificial Sequence
  Synthetic polynucleotide
aaggtgaaggtcggagtc

SEQ ID NO: 4 - DNA, 18 bp, Artificial Sequence
  Synthetic polynucleotide
gattttggagggatctcg

SEQ ID NO: 5 - DNA, 20 bp, Artificial Sequence
  Synthetic polynucleotide
gcttcgggagccctctggga

SEQ ID NO: 6 - DNA, 20 bp, Artificial Sequence
  Synthetic polynucleotide
tgagcgagtagccggacccc

SEQ ID NO: 7 - DNA, 20 bp, Artificial Sequence
  Synthetic polynucleotide
gcttcgggagccctctggga

SEQ ID NO: 8 - DNA, 20 bp, Artificial Sequence
  Synthetic polynucleotide
tgagcgagtagccggacccc

SEQ ID NO: 9 - DNA, 19 bp, Artificial Sequence
  Synthetic polynucleotide
ccacagtccatgccatcac

SEQ ID NO: 10 - DNA, 19 bp, Artificial Sequence
  Synthetic polynucleotide
tccaccaccctgttgctgt

SEQ ID NO: 11 - protein, 25 aa, Artificial Sequence
  Synthetic polypeptide
EYWLGNEFVSQLTNQQRYVLKIHLK

SEQ ID NO: 12 - protein, 20 aa, Artificial Sequence
  Synthetic polypeptide
HTTNGIYTLTFPNSTEEIKA

SEQ ID NO: 13 - protein, 15 aa, Artificial Sequence
  Synthetic polypeptide
WKGSGYSLKATTMMI

SEQ ID NO: 14 - protein, 15 aa, Artificial Sequence
  Synthetic polypeptide
EYWLGNEFVSQLTNQ

SEQ ID NO: 15 - protein, 15 aa, Artificial Sequence
  Synthetic polypeptide
HTTNGIYTLTFPNST

SEQ ID NO: 16 - protein, 15 aa, Artificial Sequence
  Synthetic polypeptide
LNYRIHLKGLTGTAG

SEQ ID NO: 17 - protein, 15 aa, Artificial Sequence
  Synthetic polypeptide
IYTLTFPNSTEEIKA

SEQ ID NO: 18 - protein, 15 aa, Artificial Sequence
  Synthetic polypeptide
NEFVSQLTNQQRYVL

SEQ ID NO: 19 - protein, 15 aa, Artificial Sequence
  Synthetic polypeptide
QLTNQQRYVLKIHLK

(19) United States
(12) Patent Application Publication
(10) Pub. No.: US 2016/0215289 A1
(43) Pub. Date: Jul. 28, 2016

(54) METHODS OF MODULATING LYMPHANGIOGENESIS, E.G., TO TREAT CORNEAL TRANSPLANT REJECTION, IN A SUBJECT

(71) Applicant: The Regents of the University of California, Oakland, CA (US)

(72) Inventor: Lu Chen, Berkeley, CA (US)

(21) Appl. No.: 15/004,646

(22) Filed: Jan. 22, 2016

Related U.S. Application Data
(60) Provisional application No. 62/106,418, filed on Jan. 22, 2015.

(51) Int. Cl. C12N 15/113; A61K 45/06; A61K 31/713

(52) U.S. Cl. CPC C12N 15/1136; A61K 31/713; A61K 45/06; C12N 2310/14; C12N 2320/30

(57) ABSTRACT

Methods of modulating the occurrence of lymphangiogenesis in a subject are provided. In some instances, the method includes reducing antigen presenting cell function, such as cell trafficking to draining lymph nodes. In some instances, the method includes treating corneal transplant rejection in the subject. Aspects of the methods include administering to the subject an effective amount of an antagonist of angiopoietin-2 (Ang-2) to reduce the occurrence of lymphangiogenesis. In some cases, the method results in the improvement of graft survival in the subject. Also provided are methods of reducing immune cell activity in a sample, e.g., reducing antigen presenting cell trafficking to draining lymph nodes. Also provided are compositions, e.g., ophthalmic pharmaceutical compositions and kits that find use in the subject methods.

### CROSS-REFERENCE TO RELATED APPLICATIONS

Pursuant to 35 U.S.C. §119 (e), this application claims priority to the filing date of the U.S. Provisional Application No. 62/106,418, filed on Jan. 22, 2015, the disclosure of which application is herein incorporated by reference in its entirety.

### STATEMENT REGARDING FEDERALLY SPONSORED RESEARCH

This invention was made with government support under contract EY017392 awarded by the National Institutes of Health. The government has certain rights in the invention.

### INTRODUCTION

Accompanying the blood circulation, the lymphatic vascular network penetrates most tissues in the body and plays important roles in a broad spectrum of functions, including immune surveillance, fat absorption, and interstitial fluid homeostasis. Numerous disorders are associated with lymphatic dysfunction, such as cancer metastasis, inflammatory and immune diseases, transplant rejection, and lymphedema. Lymphatic endothelial molecular markers that find use in investigating lymphatic tissues and pathologic lymphatic processes, such as lymphangiogenesis (LG), include vascular endothelial growth factor receptor-3 (VEGFR-3), lymphatic vessel endothelial hyaluronan receptor-1 (LYVE-1) and Prox-1.

Lymphangiogenesis (LG) is involved in a number of diseases, such as cancer metastasis, inflammatory diseases and immune diseases. For example, one reason for transplant failure is immune-mediated rejection. Transplantation can restore the functions of a tissue or an organ to patients when other treatments have failed or the patients are experiencing medical emergencies. Corneal transplantation is an example of a tissue or solid organ transplantation. Patients who are blind as a result of corneal diseases after traumatic, inflammatory, infectious, or chemical injuries tend to have inflamed and vascularized corneas and experience a transplant rejection rate which can be as high as 90%.

### SUMMARY

Methods of modulating the occurrence of lymphangiogenesis in a subject are provided. In some instances, the method includes reducing antigen presenting cell function, such as cell trafficking to draining lymph nodes. In some instances, the method includes treating corneal transplant rejection in the subject. Aspects of the methods include administering to the subject an effective amount of an antagonist of angiopoietin-2 (Ang-2) to reduce the occurrence of lymphangiogenesis. In some cases, the method is a method of improving graft survival in the subject. Also provided are methods of reducing immune cell activity in a sample, e.g., reducing antigen presenting cell trafficking to draining lymph nodes. Also provided are compositions, e.g., ophthalmic pharmaceutical compositions and kits that find use in the subject methods.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1, Panels A-C illustrate that angiopoietin-2 (Ang-2) is expressed on lymphatic vessels and macrophages in inflamed cornea. (Panel A) Representative images of immunofluorescent microscopic assays showing Ang-2 (green) coexpressed with LYVE-1+ (red) lymphatic vessels in the inflamed cornea. Scale bar: 100 (Panel B) Representative images showing Ang-2 (red) expression on F4/80+ (green) macrophages in the inflamed cornea. Scale bar: 10 μm. (Panel C) Quantitative PCR analysis showing significant increase of Ang-2 expression in corneal macrophages in the inflamed condition. *P<0.05.

FIG. 2, Panels A-C illustrate that corneal LG response is inhibited in Ang-2 knockout mice. (Panel A) Angiopoietin-2 knockout mice showing swollen hind paws reflecting lymphedema. (Panel B) Representative images of immunofluorescent microscopic assays showing significantly reduced lymphatic vessels (LYVE-1+, red) in the inflamed cornea of Ang-2 knockout mice compared with wild-type controls. Corneas were harvested 14 days after suture placement. White dotted line: demarcation between the cornea and conjunctiva. Scale bars: 200 μm. (Panel C) Summarized data from repetitive experiments showing significant difference in lymphatic invasion area between the two groups. ***P<0.001.

FIG. 3, Panels A-C show abnormal patterning of blood vessels in Ang-2 knockout mice. (Panel A) Representative images from ophthalmic slit-lamp bioscopy showing disorganized and shortened blood vessels in Ang-2 knockout mice. White dotted line: demarcation between the cornea and conjunctiva. Scale bar: 350 μm. (Panel B) Representative images of immunofluorescent microscopic assays showing difference of blood vessels in inflamed corneas of Ang-2 and wild-type control mice. Scale bar: 250 μm. Bottom panels: higher magnification view of boxed areas in the upper panels showing an increase in branching points and a decrease in diameters of blood vessels in Ang-2 knockout mice. Scale bar: 100 μm. (Panel C) Summarized data from repetitive experiments showing the differences of blood vessels in Ang-2 knockout and wild-type mice in terms of invasion area, branching point, and diameter. *P<0.05. **P<0.01.

FIG. 4, Panels A-F illustrate angiopoietin-2 expression and depletion in LECs. (Panels A, B) Reverse transcription-PCR analysis and immuncytofluorescent microscopic analysis of Ang-2 expression in human microdermal LECs. Angiopoietin-2: red. DAPI nuclear staining: blue. Scale bar: 50 μm. (Panel C) Reverse transcription-PCR assay showing Ang-2 gene depletion in LECs 48 hours following transfection with Ang-2 siRNA, as compared with scrambled siRNA. (Panel D) Summarized data showing significant inhibition of LEC proliferation following transfection with Ang-2 siRNA, as revealed by MTS proliferation assay. ***P<0.001. (Panel E) Representative micrographs showing significant suppression of LEC capillary tube formation in matrigel following transfection with Ang-2 siRNA. Scale bars: 100 μm. (Panel F) Summarized data on total tubule length measurement. **P<0.01.

FIG. 5, Panels A-B demonstrate corneal LG response is inhibited by anti-Ang-2 treatment. (Panel A) Representative images of immunofluorescent microscopic analysis showing significantly reduced lymphatic vessels (LYVE-1+, red) in the inflamed cornea of Ang-2 siRNA treatment group. White dotted line: demarcation between the cornea and conjunctiva. Scale bar: 200 μm. (Panel B) Summarized data from repetitive experiments showing significant difference in lymphatic invasion area. **P<0.01.

FIG. 6, Panels A-C illustrate anti-Ang-2 antibody treatment suppresses corneal lymphangiogenesis and antigen presenting cell trafficking to draining lymph nodes after transplantation and promotes corneal graft survival in a low risk mouse model of corneal transplantation. Panel A: whole mount corneal images showing reduced lymphatic vessels (green) in the treatment group; Panel B: flow cytometry analysis showing reduced antigen presenting cell trafficking to draining lymph nodes in the treatment group; Panel C: Kaplan-Meier survival curves showing increased graft survival in the treatment group.

### DETAILED DESCRIPTION

As summarized above, aspects of the present disclosure include methods of modulating the occurrence of lymphangiogenesis in a subject. In some instances, the method is a method of treating corneal transplant rejection in the subject. In some instances, the method is a method of modulating antigen presenting cell trafficking to draining lymph nodes. Aspects of the methods include administering to the subject an effective amount of an Ang-2 antagonist to reduce the occurrence of lymphangiogenesis, e.g., by inhibiting the trafficking of antigen presenting cell to draining lymph nodes or improving transplant survival in the subject. Also provided are methods of reducing immune cell activity in a sample, e.g., reducing antigen presenting cell trafficking to draining lymph nodes. Also provided are compositions, e.g., ophthalmic pharmaceutical compositions and kits, etc., that find use in the subject methods. Embodiments of the present disclosure find use in a variety of different applications, including research and therapeutic applications.

Before the present invention is described in greater detail, it is to be understood that aspects of the present disclosure are not limited to the particular embodiments described, and as such may, of course, vary. It is also to be understood that the terminology used herein is for the purpose of describing particular embodiments only, and is not intended to be limiting, since the scope of embodiments of the present disclosure will be defined only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within embodiments of the present disclosure, subject to any specifically excluded limit in the stated range. Where the stated range includes one or both of the limits, ranges excluding either or both of those included limits are also included in embodiments of the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of embodiments of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that embodiments of the present disclosure are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods of Modulating Lymphangiogenesis

As summarized above, aspects of the present disclosure include methods of modulating the occurrence of lymphangiogenesis in a subject including administering an effective amount of an angiopoietin-2 (Ang-2) antagonist to the subject.

Angiopoietin-2 is a ligand belonging to the angiopoietin-Tie family that is a regulatory molecule in angiogenesis and also modulates lymphangiogenesis. Ang-2's functions through the Tie-2 receptor are dependent on the cellular textures. Ang-2 is expressed during lymphatic development at embryonic and neonatal stages, and Ang-2 knockout (KO) mice demonstrate severe defects in lymphatic patterning and clinical signs of chylous ascites and lymphedema. However, the specific roles of Ang-2 in pathologic lymphatic processes have remained largely unknown.

Using an in vivo corneal inflammatory LG model, an in vitro human skin lymphatic endothelial cell (LEC) culture system, and an in vivo corneal transplantation model, the inventors have shown that: (1) Ang-2 is expressed on both lymphatic vessels and macrophages in inflamed cornea; (2) corneal LG response is almost abolished in inflamed cornea of Ang-2 gene knockout mice while the hemangiogenesis (HG) response is significantly suppressed with disorganized blood vessels; (3) Ang-2 gene knockdown by siRNAs markedly inhibits LEC functions of proliferation and tube formation in vitro; (4) treatment with a Ang-2 antagonist (e.g., siRNAs) is effective in suppressing corneal LG in vivo, and (5) treatment with a Ang-2 antagonist (e.g., an anti-Ang2 antibody) suppresses transplantation associated corneal LG in recipient beds, and suppresses antigen presenting cell trafficking to draining lymph nodes after transplantation, thereby promoting corneal graft survival and reducing transplant rejection. These data together indicate that Ang-2 is critically involved in LG and transplantation immunity as well, and its manipulation offers a new therapeutic strategy to interfere with LG, immune responses, and related diseases, including transplant rejection in a variety of tissues.

Corneal transplantation is a form of solid-tissue transplantation in humans with a two-year survival rate of 90 percent on uninflamed and avascular (i.e., low-risk) corneas. However, this rate is greatly reduced to less than 50 percent on inflamed and vascularized (i.e., high-risk) corneas. Unfortunately, many patients who are blind as a result of corneal diseases fall in this high-rejection category. In certain embodiments, the subject methods provide for modulation (e.g., reduction) of corneal inflammatory LG in a subject in need of, or undergoing, a corneal transplant, where the subject can have a high risk or a low risk of transplant rejection. In some instances, the method provides for modulation (e.g., reduction) of antigen presenting cell trafficking in the subject.

As the forefront medium in the passage of light to the retina, the cornea by nature maintains transparency and is normally devoid of any vasculatures. The cornea is also different from other tissues in part because it is immune privileged. The presence, longevity, and importance of lymphatic vessels in the cornea under pathological situations were questioned for a long time. Lymphatics are induced in the cornea after inflammatory, infectious, traumatic, chemical or toxic insults. Corneal lymphatics can be induced independently of blood vessels. The lymphatic pathway is involved in the induction of corneal transplantation immunity. The molecular mechanisms of corneal LG in corneal transplantation are more complex than previously considered. A variety of factors are involved in lymphatic processes and the promotion of corneal transplant survival. For example, Ang-2 plays differential roles in corneal LG versus HG.

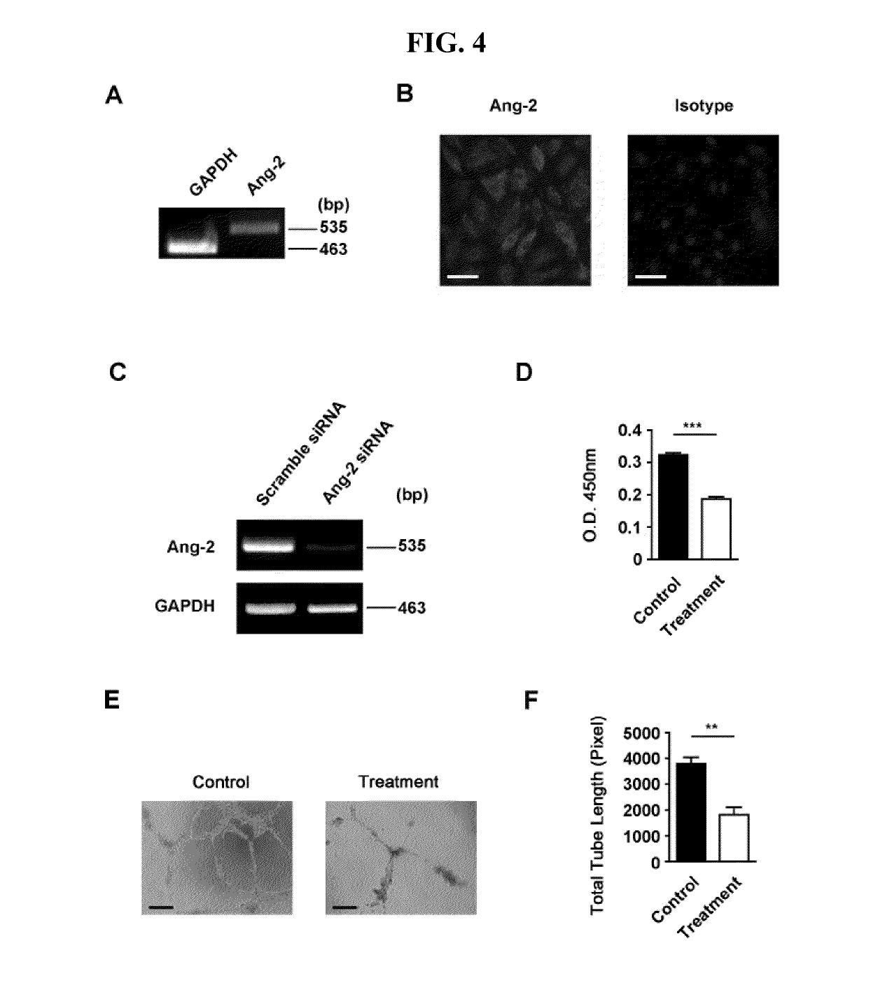

Lymphatic endothelial cells are cells which make up the lymphatic vessels and capillaries of the lymph system. FIG. 4 illustrates the inhibition of human skin LEC proliferation and the suppression of LEC capillary tube formation following contacting of the cells with an exemplary Ang-2 antagonist (e.g., an Ang-2 siRNA agent). As such, aspects of the present disclosure include a method including contacting a sample including a cell or tissue of interest with an effective amount of an Ang-2 antagonist (e.g., as described herein), where the method can be performed in vitro or in vivo. Any convenient cells or tissues can be targeted for use in the subject methods. In some cases, the cell or tissue of interest is one which includes lymphatic endothelial cells. In certain instances, the method is a method of inhibiting lymphatic endothelial cell (LEC) proliferation. In some embodiments, the method is a method of inhibiting tube formation. In certain instances, the method is a method of inhibiting immune cell function, e.g., an antigen presenting cell trafficking to draining lymph nodes. As such, in some instances, the method is a method of suppressing immune system reaction to foreign body (e.g., transplanted tissue). Any convenient samples where it is desirable to modulate the activity of immune cells of interest can be utilized in the subject methods. In certain embodiments, the context in which the immune cells are present does not include LG activity. In certain instances, the immune cells are antigen presenting cells. The tissue can comprise a graft bed and/or a transplanted tissue. Any convenient tissues can be utilized, including but not limited to, heart tissue, lung tissue, kidney tissue, skin tissue or ocular tissue. In certain embodiments, the tissue is corneal tissue. In certain embodiments, the method inhibits capillary tube formation. In certain embodiments, the method inhibits corneal LG. In certain embodiments, contacting the cell with an Ang-2 antagonist inhibits cell chemotaxis. In some cases, the method is a method of inhibiting cell chemotaxis including contacting a sample comprising the cell with a Ang-2 antagonist (e.g., as described herein).

As used herein, the term “lymphangiogenesis (LG)” refers to the growth of new lymphatic vessels. LGcan occur at a variety of sites in the subject. LG can be involved in a variety of pathological or disease conditions including neoplasm metastasis, edema, rheumatoid arthritis, psoriasis, lymphangiomatosis and impaired wound healing. In some cases, lymphangiogenesis is inflammatory. In some cases, modulating the occurrence of lymphangiogenesis means the growth of new lymphatic vessels is at least ameliorated. As used herein, the terms ameliorate and amelioration are used in a broad sense to refer to at least a reduction in the magnitude of a parameter, e.g. symptom, associated with the condition being targeted.

In some instances, modulating the occurrence of lymphangiogenesis is meant to encompass the amelioration of one or more functions of lymphatic vessels associated with a pathologic or disease condition. In certain embodiments, the one or more functions of lymphatic vessels which are at least ameliorated include, but are not limited to, transport of lymph through the vessels, transport of any convenient components of the lymphatic system (e.g., antigens and/or antigen-presenting cells) through the afferent pathway, draining of lymph nodes; infiltrations of macrophages, leukocytes, or T cells; and acting as reservoir for lymphatic system components (e.g., as described herein). Such functions of the lymphatic vessels can be measured using any convenient bioassay, or in vivo.

In certain cases, the parameter of interest, e.g., growth and/or function of lymphatic vessels, is reduced or inhibited by about 20% or more, such as by about 30% or more, about 40% or more, about 50% or more, about 60% or more, about 70% or more, about 80% or more, about 90% or more, or about 100% as compared to a suitable control, and as measured by a convenient bioassay, or in vivo.

In some instances, prior to the administrating step, lymphangiogenesis has occurred in the subject, and the method of modulating includes reducing lymphangiogenesis in the subject. In certain instances, lymphangiogenesis has not occurred in the subject prior to the administrating step, and the method of modulating includes preventing lymphangiogenesis from occurring. In such cases, the subject can be at risk of experiencing lymphangiogenesis, but this risk is reduced by practicing the subject methods. In some embodiments, modulating the occurrence of lymphangiogenesis means that lymphangiogenesis does not occur in the subject, e.g., lymphangiogenesis is prevented. In other words, practicing the subject method includes maintaining the tissue of the subject in a lymphatic-free or low state.

The methods of the present disclosure find use in the treatment of a variety of conditions associated with lymphangiogenesis. Pathology or disease conditions of interest include, but are not limited to, cancers, infections, inflammatory conditions, neoplasm metastasis, edema, rheumatoid arthritis, psoriasis, lymphangiomatosis, impaired wound healing and transplant rejections. In some cases, the methods find use in treatment of patients in need of a transplant, including those patients at risk of transplant rejection. As such, in certain instances, the method further includes transplanting tissue in a graft bed of the subject. As such, in certain cases, the method further includes transplanting an organ into the subject.

Lymphangiogenesis, once induced, is a primary mediator of transplant rejection. The clinical burden of graft rejection in the high-risk transplantation is tremendous, since as high as e.g., 50-90% of the grafts are rejected irrespective of current treatment modalities.

In certain embodiments, lymphangiogenesis is suppressed by at least 10%. In certain embodiments, lymphangiogenesis is suppressed by at least 10%, such as at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or even 100%. In certain embodiments, lymphangiogenesis is suppressed at a level sufficient to cause a therapeutic effect. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the condition. Therapeutic effects can be measured quantitatively by a physician or qualitatively by a patient afflicted with LG targeted by the therapeutic agent (e.g., siRNA).

In certain embodiments, antigen presenting cell trafficking is suppressed by at least 10%, such as at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or even 100%, as compared to a suitable control. In certain embodiments, antigen presenting cell trafficking is suppressed at a level sufficient to cause a therapeutic effect, e.g., modulation of an undesirable immune reaction to transplanted tissue.

In some embodiments, the method includes enhancing survival of the transplanted tissue in a subject. In some embodiments, the method includes enhancing survival of the transplanted organ in a subject. As used herein, by “enhancing survival” is meant an increase in transplant survival rate as measured at any convenient time post transplantation, using any convenient bioassay, or in vivo. In some instances, the survival rate is enhanced to a rate of about 20% or more, about 30% or more, about 30% or more, about 40% or more, about 50% or more, about 60% or more, about 70% or more, about 80% or more, about 90% or more, or about 100% as measured in a convenient bioassay, or in vivo, as compared to a suitable control. In some instances, where the transplanted tissue is ocular tissue, and the survival rate is measured at a convenient time 2 or more weeks post-transplantation, such as, at 3, 4, 5, 6, 7 or 8 weeks post-transplantation, the survival rate is enhanced to a rate of about 20% or more, such as by about 30% or more, about 40% or more, about 50% or more, about 60% or more, about 70% or more, about 80% or more, about 90% or more, or about 100% as measured in a convenient bioassay, or in vivo. In certain embodiments, the negative control survival rate of transplantation is about 50% or less, such as about 40% or less, about 30% or less, or 20% or less.

Prior to transplantation, the graft bed can be inflamed and vascularized, and administering the Ang-2 antagonist can inhibit lymphatic vessel growth and/or function to reduce inflammatory lymphangiogenesis in the graft bed. In some embodiments, the graft bed is uninflamed and avascular and the method includes maintaining the graft bed as uninflamed and avascular before, during and/or after transplantation. Administration of the Ang-2 antagonist can result in selective inhibition of the growth of and/or a function of the lymphatic vessels while having no significant effect on the growth of and/or a function of the blood vessels.

Practicing the subject methods can result in selective inhibition of the growth of lymphatic vessels of interest relative to the growth of blood vessels of interest. In certain embodiments, the subject methods can result in selective inhibition of one or more functions of the lymphatic vessels (e.g., as described herein) relative to one or more analogous functions of the blood vessels. By selective inhibition is meant the lymphatic vessels are inhibited to a greater extent than the blood vessels. In certain cases, blood vessel growth and/or function is not significantly inhibited (In certain cases, blood vessel growth and/or function is inhibited by about 20% or less, such as 10% or less, or 5% or less. In certain cases, lymphatic vessel growth and/or function is inhibited in the corneal tissue. It is understood that the selection of lymphatic and blood vessels of interest for determining selective inhibition is dependent on a number of factors, such as the type of tissue involved, the type of pathologic insult to the tissue, the site and location of a graft bed, and the site of administration, which factors can be readily determined.

In some cases, the lymphatic vessels are inhibited (e.g., lymphatic vessel growth and/or function) by about 30% or more, while the blood vessels are inhibited (e.g., blood vessel growth and/or function) by about 30% or less. In certain instances, the lymphatic vessels are inhibited by about 30% or more, such as about 40% or more, about 50% or more, about 60% or more, about 70% or more, about 80% or more, about 90% or more, or about 100% as measured by any convenient bioassay, or in vivo. In certain embodiments, the blood vessels are inhibited by about 70% or less, such as about 60% or less, about 50% or less, about 40% or less, about 30% or less, about 20% or less, about 10% or less, about 5% or less, or does not inhibit at all. All of the above values are presented in terms of a suitable control.

In some cases, the method modulates lymphangiogenesis of the corneal tissue. In certain embodiments, inflammatory lymphangiogenesis of the corneal tissue is eliminated, e.g., the tissue is transformed from an inflamed lymphangiogenicstate to an uninflamed lymphatic-reduced state. In some embodiments, the untreated corneal tissue is inflamed and vascularized, and after treatment according to the subject methods the tissue is reduced in inflammation and vascularity, as measured in a convenient bioassay, or in vivo. In certain cases, after treatment, the tissue is uninflamed and/or avascular. In some instances, practicing the subject method reduces the occurrence of opacity of the corneal tissue. In some embodiments, the opacity of the corneal tissue is reduced by about 20% or more, such as about 30% or more, about 40% or more, about 50% or more, about 60% or more, about 70% or more, about 80% or more, about 90% or more, or about 100%, as measured in a convenient bioassay, or in vivo, at a convenient time post-transplantation, e.g., 2 or more weeks, such as 3, 4, 5, 6, 7 or 8 weeks post-transplantation, as compared to a suitable control.

As used herein, the term “effective amount” refers to that amount of a substance (e.g., an antagonist of interest) that produces some desired local or systemic effect. Effective amounts of active agents of interest vary depending on a variety of factors including, but not limited to, the weight and age of the subject, the condition being treated, the severity of the condition, the manner of administration and the like, and can readily be determined, e.g., determined empirically using data such as that data provided in the experimental section below.

### Ang-2 Antagonists

Aspects of the methods include the use of a modulator of Ang-2 activity. The modulator may, in some instances, be an antagonist of Ang-2 activity. As used herein, the term “antagonist” refers to any agent that blocks or reduces a biological activity mediated by Ang-2. In some cases, the antagonist blocks a biological response of the target that is mediated by the target agonist. In other words, the antagonist reduces or inhibits the activation of the target by the agonist. The antagonist can act directly or indirectly. The antagonist can act directly by disrupting the binding of an agonist to the active site of the target. The antagonist can mediate its blocking effect by binding to the active site of the target, or to an allosteric site of the target, or to any other convenient site of the target. In some embodiments, the antagonist is an inhibitor that competes with one or more endogenous ligands or substrates of the target. In some embodiments, an antagonist is an agent that interferes with one or more receptor-ligand binding interactions. In certain cases, the antagonist inhibits receptor signal transduction. The antagonist can act indirectly via blocking the action of a co-factor involved in biological regulation of the receptor's activity. The antagonist can act indirectly by blocking or inhibiting expression of the receptor, or an endogenous ligand or substrate thereof. Antagonist activity can be reversible or irreversible.

Any convenient agents can be utilized as an antagonist of Ang-2 in the subject methods and compositions. In some embodiments, the agent is an agent that modulates, e.g., inhibits, Ang-2 activity by binding directly to Ang-2. In certain embodiments, the administered active agent is an Ang-2 specific binding member. In general, useful Ang-2 specific binding members exhibit an affinity (Kd) for a target Ang-2, such as human Ang-2, that is sufficient to provide for the desired reduction in occurrence of lymphangiogenesis. As used herein, the term “affinity” refers to the equilibrium constant for the reversible binding of two agents; “affinity” can be expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of a specific binding member to a target protein can be, for example, from about 100 nanomolar (nM) to about 0.1 nM, from about 100 nM to about 1 picomolar (pM), or from about 100 nM to about 1 femtomolar (fM) or more. The term “binding” refers to a direct association between two molecules, due to, for example, covalent, electrostatic, hydrophobic, and ionic and/or hydrogen-bond interactions, including interactions such as salt bridges and water bridges. In some embodiments, the agent binds human Ang-2 with nanomolar affinity or picomolar affinity. In some embodiments, the agent binds human Ang-2 with a Kd of less than about 100 nM, less than about 50 nM, less than about 20 nM, less than about 10 nM, or less than about 1 nM. In some embodiments, the affinity between the binding member active agent in a binding complex with Ang-2 is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

Agents of interest include, but are not limited to, a ligand, a receptor, an Ang-2-binding antibody, a scaffolded protein binder of Ang-2, a nucleic acid, a small molecule, and a peptide; or a fragment, variant, or derivative thereof; or combinations of any of the foregoing.

Antibodies that can be used as antagonists in connection with the present disclosure can encompass, but are not limited to, monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments and dsFv antibody fragments. Furthermore, the antibody molecules can be fully human antibodies, humanized antibodies, or chimeric antibodies. The antibodies that can be used in connection with the present disclosure can include any antibody variable region, mature or unprocessed, linked to any immunoglobulin constant region. Minor variations in the amino acid sequences of antibodies or immunoglobulin molecules are encompassed by the present disclosure, providing that the variations in the amino acid sequence maintain 75% or more, e.g., 80% or more, 90% or more, 95% or more, or 99% or more of the sequence. In particular, conservative amino acid replacements are contemplated. Conservative replacements are those that take place within a family of amino acids that are related in their side chains. Whether an amino acid change results in a functional peptide can be determined by assaying the specific activity of the polypeptide derivative.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that can be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. In some embodiments, the antibody molecules are monoclonal, fully human antibodies.

The antibodies that can be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some embodiments, fully human monoclonal antibodies directed against Ang-2 are generated using transgenic mice carrying parts of the human immune system rather than the mouse system.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations can be used to define structural and functional domains in accordance with the invention.

Specific examples of antibody agents that can be employed as Ang-2 antagonist include, but are not limited to, the human monoclonal antibody MEDI-3617, the human monoclonal antibody 3.19.3 (Brown et al., Mol. Cancer Ther., 2010, 9(1), 145-56), human antibodies LC06 and LC08 (Thomas et al., PLoS One. 2013; 8(2):e54923), fully human anti-Ang-2 antibody REGN-910, AMG-780 and those described in, US20150337033, US20150079112, US20130156789, US20110311546 and US20110236388; the disclosures of which are herein incorporated by reference. Examples of a bispecific antibodies of interest include but are not limited to, CovX-Bodies such as CVX-060 (PF04856884), and the like.

In some instances, the antagonist is an antibody, an Ang-2-binding fragment or domain thereof, or a derivative thereof. The antibody can be used to decrease Ang-2 activity in a tissue of interest. Non-limiting examples of such antibodies include antibodies directed against any suitable epitope of Ang-2; and antibodies directed against a soluble Ang-2 ligand or a Ang-2-ligand complex. Also encompassed are bi-specific antibodies, e.g., antibodies in which each of the two binding domains recognizes a different binding epitope. In some embodiments, antibodies include those that reduce the interaction between the Ang-2 and one or more ligands. Such blocking antibodies can be identified using any convenient competition assays. In certain embodiments, the Ang-2 specific antibody is configured to inhibit signaling.

In some cases, the antagonist is a ligand of Ang-2. Such ligands can include a structural modification relative to an endogenous ligand that renders the modified ligand effective in inhibiting the activity of Ang-2. In certain cases, such Ang-2-binding antagonists are selected from polypeptides, nucleic acids, small molecules, and analogs or derivatives thereof.

In certain cases, the antagonist is a soluble polypeptide. A variety of soluble polypeptides can be used as antagonists of Ang-2. Such soluble polypeptide antagonists include, but are not limited to, a polypeptide that includes a domain of the Ang-2 protein, or a convenient analog or fragment thereof. Ang-2 domains of interest include, but are not limited to, the Ang-2 receptor binding domain, N-terminal super clustering domain, the central coiled domain, the linker region and the C-terminal fibrinogen-related domain. Such soluble polypeptides can bind with high affinity to an endogenous ligand of the Ang-2 target. As used herein, soluble polypeptides include fragments, functional variants, and modified forms of the soluble polypeptides. These fragments, functional variants, and modified forms of soluble polypeptides can antagonize the function of the Ang-2. Isolated fragments of these soluble polypeptides can be obtained by screening polypeptides recombinantly produced from the corresponding fragment of the nucleic acid encoding the soluble polypeptide. In addition, fragments can be chemically synthesized using any convenient techniques, e.g., conventional solid phase peptide synthesis using Fmoc or Boc chemistry. The fragments can be produced (recombinantly or by chemical synthesis) and tested to identify those peptidic fragments that can function to inhibit function of the Ang-2. In certain embodiments, the antagonist is a functional variant of a soluble polypeptide comprising an amino acid sequence that is 80% or more, such as 85% or more, 90% or more, 95% or more, 97% or more, or 99% or more identical to a domain of the Ang-2 (e.g., as described herein).

Examples of polypeptides of interest which find use in the subject methods and compositions to inhibit the activity Ang-2, include but are not limited to, those polypeptides described by US20140113858, WO2004/092215 and WO03/030833, the disclosures of which are herein incorporated by reference. In certain instances, the peptide of interest is at least one selected from: a peptide comprising 5 to 25 consecutive amino acids of SEQ ID NO: 11 (EYWLGNEFVSQLTNQQRYVLKIHLK), including the amino acid at position 11 (Q) of SEQ ID NO: 11, a peptide comprising 11 to 20 consecutive amino acids of SEQ ID NO: 12 (HTTNGIYTLTFPNSTEEIKA), including amino acids from 6th to 15th positions of SEQ ID NO: 12, a peptide comprising the amino acid sequence of SEQ ID NO: 13 (WKGSGYSLKA TTMMI); a peptide comprising the amino acid sequence of SEQ ID NO: 14 (EYWLGNEFVS QLTNQ), a peptide comprising the amino acid sequence of SEQ ID NO: 15 (HTTNGIYTLT FPNST), a peptide comprising the amino acid sequence of SEQ ID NO: 16 (LNYRIHLKGL TGTAG), a peptide comprising the amino acid sequence of SEQ ID NO: 17 (IYTLTFPNST EEIKA), a peptide comprising the amino acid sequence of SEQ ID NO: 18 (NEFVSQLTNQ QRYVL), and a peptide comprising the amino acid sequence of SEQ ID NO: 19 (QLTNQQRYVL KIHLK).

In some embodiments, the antagonist is a scaffolded polypeptide binder. A scaffold refers to an underlying peptidic framework (e.g., a consensus sequence or structural motif) from which a polypeptide agent arose, e.g., via phage display screening of a polypeptide library, or from a chimeric protein construct. The underlying scaffold sequence includes those residues that are fixed and variant residues that can confer on the resulting polypeptide agents different functions, such as specific binding to a target receptor. Such structural motifs can be characterized and compared structurally as a combination of particular secondary and tertiary structural elements, or alternatively, as a comparable primary sequence of amino acid residues. Any convenient scaffolds and scaffolded polypeptides can be utilized as antagonists in the subject methods. In some embodiments, such antagonists can be identified utilizing a recombinant screening method such as phage display screening. Scaffolded polypeptide binders of interest include, but are not limited to, synthetic small proteins and recombinant small proteins such as Affibodies. Examples of scaffolded polypetides include, but are not limited to, peptide-Fc fusion proteins which bind Ang-2, such as L1-10 (Suzuki, R. et al. (2013) Inhibition of angiopoietin 2 attenuates lumen formation of tumour-associated vessels in vivo. International Journal of Oncology 43(5). DOI: 10.3892/ijo.2013.2076), L1-7(N) (Hashizume et al., (2010) Complementary actions of inhibitors of angiopoietin-2 and VEGF on tumor angiogenesis and growth. Cancer Res 70: 2213-2223), Trebananib (AMG-386) (Gerald, D. et al. (2013) Angiopoietin-2: An Attractive Target for Improved Antiangiogenic Tumor Therapy. Cancer Res 73: 1649), and the like. In certain instances, the Ang-2 antagonist is not L1-10. In certain instances, the Ang-2 antagonist is not L1-7(N). In certain instances, the Ang-2 antagonist is not trebananib.

In some cases, the antagonist is an aptamer nucleic acid molecule or analog thereof which directly binds, e.g., to Ang-2 or an Ang-2 ligand or receptor. Aptamers of interest include, but are not limited to, aptamer 11-1.41 RNA described by White R R, et al. ((2003) Inhibition of rat corneal angiogenesis by a nuclease-resistant RNA aptamer specific for angiopoietin-2. Proc Natl Acad Sci USA 100(9):5028-5033), the disclosure of which is herein incorporated by reference.

Ang-2 specific binding members also include non-antibody binding members. For example, small molecules that bind to Ang-2 and inhibit its activity are of interest. In some instances, the antagonist is a small molecule antagonist of Ang-2. Small molecules of interest include, but are not limited to, small organic or inorganic compounds having a molecular weight (MW) of more than 50 and less than about 2,500 daltons (Da), such as more than 50 and less than about 1000 Da, or more than 50 and less than about 500 Da. “Small molecules” encompasses numerous biological and chemical classes, including synthetic, semi-synthetic, or naturally-occurring inorganic or organic molecules, including synthetic, recombinant or naturally-occurring polypeptides and nucleic acids. Small molecules of interest can comprise functional groups necessary for structural interaction with proteins, particularly hydrogen bonding, and can include at least an amine, carbonyl, hydroxyl or carboxyl group, and can contain at least two of the functional chemical groups. The small molecules can comprise cyclical carbon or heterocyclic structures and/or aromatic or polyaromatic structures substituted with one or more of the above functional groups. Small molecules are also found among biomolecules including peptides, saccharides, fatty acids, steroids, purines, pyrimidines, derivatives, structural analogs or combinations thereof.

Agents finding use in the subject methods also include agents that modulate expression of the RNA and/or protein from the Ang-2 gene, such that it changes the expression of the RNA or protein from the target Ang-2 gene in some manner. In these instances, the agent can change expression of the RNA or protein in a number of different ways. In certain embodiments, the agent is one that reduces, including inhibits, expression of an Ang-2 protein. Inhibition of Ang-2 protein expression can be accomplished using any convenient protocol, including use of an agent that inhibits Ang-2 protein expression, such as, but not limited to: RNAi agents, antisense agents, agents that interfere with a transcription factor binding to a promoter sequence of the Ang-2 gene, or inactivation of the Ang-2 gene, e.g., through recombinant techniques, etc. In some cases, the antagonist is an inhibitor of expression of the Ang-2 or its receptor, or an endogenous ligand of Ang-2. In certain instances, the antagonist inhibits expression of Ang-2 itself. In certain instances, the antagonist modulates expression of the receptor tyrosine kinase Tie-2. Any convenient inhibitor of expression can be utilized as an antagonist in the subject methods. Such antagonists can act to inhibit expression at a transcriptional, translational, or post-translational level. In some embodiments, the inhibitors are nucleic-acid based, including, without limitation, DNA, RNA, chimeric RNA/DNA, protein nucleic acid, and other nucleic acid derivatives. In some embodiments, the expression inhibitors encompass RNA molecules capable of inhibiting receptor production when introduced into a receptor-expressing cell (termed RNAi), including short hairpin double-stranded RNA (shRNA). In some instances, the expression inhibitors are small interfering RNA (siRNA). It will be understood that any sequence capable of reducing the cell surface expression of a receptor, or reducing the expression of a receptor ligand, can be used in practicing the methods of the present disclosure.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The disclosure encompasses isolated or substantially purified nucleic acid nucleic acid molecules and compositions containing those molecules. In the context of the present disclosure, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present disclosure. By “fragment” or “portion” is meant a full length or less than full length of the nucleotide sequence. The siRNAs of the present disclosure can be generated by any method known to the art, for example, by in vitro transcription, recombinantly, or by synthetic means. In one example, the siRNAs can be generated in vitro by using a recombinant enzyme, such as T7 RNA polymerase, and DNA oligonucleotide templates.

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadelylation signal.

For example, the transcription level of an Ang-2 protein can be regulated by gene silencing using RNAi agents, e.g., double-strand RNA (see e.g., Sharp, Genes and Development (1999) 13: 139-141). RNAi, such as double-stranded RNA interference (dsRNAi) or small interfering RNA (siRNA), has been extensively documented in the nematode C. elegans (Fire, et al, Nature (1998) 391:806-811) and routinely used to “knock down” genes in various systems. RNAi agents can be dsRNA or a transcriptional template of the interfering ribonucleic acid which can be used to produce dsRNA in a cell. In these embodiments, the transcriptional template can be a DNA that encodes the interfering ribonucleic acid. Methods and procedures associated with RNAi are also described in published PCT Application Publication Nos. WO 03/010180 and WO 01/68836, the disclosures of which applications are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al., Biochem. Int. (1987) 14:1015; Bhattacharyya, Nature (1990) 343:484; and U.S. Pat. No. 5,795,715, the disclosures of which are incorporated herein by reference. Single-stranded RNA can also be produced using a combination of enzymatic and organic synthesis or by total organic synthesis. The use of synthetic chemical methods provide the introduction of desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. Development (1997)124:1133-1137; and Wianny, et al., Chromosoma (1998) 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct. Specific examples of RNAi agents that may be employed to reduce Ang-2 expression include, but are not limited to, those described by Stiehl et al. (Lung-targeted RNA interference against angiopoietin-2 ameliorates multiple organ dysfunction and death in sepsis. Crit Care Med. 2014 October; 42(10):e654-62).

In some instances, antisense molecules can be used to down-regulate expression of a Ang-2 gene in the cell. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein, and inhibits expression of the targeted protein. Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. Short oligonucleotides, of from 7 to 8 bases in length, can be strong and selective inhibitors of gene expression (see Wagner et al., Nature Biotechnol. (1996)14:840-844).

A specific region or regions of the endogenous sense strand mRNA sequence are chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra.) Oligonucleotides may be chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. Nucl. Acids Res. (1995) 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. Appl. Biochem. Biotechnol. (1995) 54:43-56.

In another embodiment, the Ang-2 gene is inactivated so that it no longer expresses a functional protein. By inactivated is meant that the gene, e.g., coding sequence and/or regulatory elements thereof, is genetically modified so that it no longer expresses a functional Ang-2 protein, e.g., at least with respect to Ang-2 activity. The alteration or mutation may take a number of different forms, e.g., through deletion of one or more nucleotide residues, through exchange of one or more nucleotide residues, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

Also of interest in certain embodiments are dominant negative mutants of Ang-2 proteins, where expression of such mutants in the cell result in a modulation, e.g., decrease, in Ang-2 activity. Dominant negative mutants of Ang-2 are mutant proteins that exhibit dominant negative Ang-2 activity. As used herein, the term “dominant-negative Ang-2 activity” or “dominant negative activity” refers to the inhibition, negation, or diminution of certain particular activities of Ang-2. Dominant negative mutations are readily generated for corresponding proteins. These may act by several different mechanisms, including mutations in a substrate-binding domain; mutations in a catalytic domain; mutations in a protein binding domain (e.g., multimer forming, effector, or activating protein binding domains); mutations in cellular localization domain, etc. A mutant polypeptide may interact with wild-type polypeptides (made from the other allele) and form a non-functional multimer. In certain embodiments, the mutant polypeptide will be overproduced. Point mutations are made that have such an effect. In addition, fusion of different polypeptides of various lengths to the terminus of a protein, or deletion of specific domains can yield dominant negative mutants. General strategies are available for making dominant negative mutants (see for example, Herskowitz, Nature (1987) 329:219, and the references cited above). Such techniques are used to create loss of function mutations, which are useful for determining protein function. Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

### Methods of Administration

Any convenient protocol for administering the Ang-2 modulator may be employed. The particular protocol that is employed may vary, e.g., depending on the site of administration and whether the antagonist is e.g., an antibody, scaffolded protein, peptide or small molecule. For in vivo protocols, any convenient administration protocol may be employed. Depending upon the identity and binding affinity of the antagonists, the response desired, the manner of administration, e.g. locally or systemic, intraocular, periocular, retrobalbar, intramuscular, intravenous, intraperitoneal, subcutaneous, subconjunctival, topical, eye drops, i.v. s.c., i.p., oral, and the like, the half-life, the number of cells, or size of the graft bed or transplanted tissue, various protocols may be employed.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the host to be treated is one amenable to treatment using the parent drug. Accordingly, a variety of subjects may be amenable to treatment using the methods and compositions disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). In some cases, the subject is a mammal that is a mouse. As used herein, the terms “host”, “subject”, and “patient” are used interchangeably.

In some instances, the subject is in need of a transplantation. In some cases, the subject may be characterized as having a high-risk of transplant rejection (e.g., the subject has a graft bed that is inflamed and vascularized). In some instances, the subject may be characterized as having a low-risk of transplant rejection (e.g., the subject has a graft bed that is uninflamed and avascular).

Also provided are in vitro methods including contacting a sample comprising cells of interest with an Ang-2 antagonist. Any convenient in vitro methods may be utilized. In some instances, the sample includes cells that are maintained in a suitable culture medium, and the antagonist is introduced into the culture medium. The term “sample” as used herein relates to a material or mixture of materials, in some cases, in fluid form, containing one or more components of interest. Samples may be derived from a variety of sources such as from a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). Components in a sample are termed “analytes” herein. In many embodiments, the sample is a complex sample containing several species of analyte.

The biological activity or response of an Ang-2 protein of interest may be mediated in vitro, in vivo, and/or in any convenient tissue or organ of interest. Tissues of interest include those tissues where blocking of Ang-2 protein is of interest. In some embodiments, the tissue comprises heart, lung, kidney, skin or ocular tissue. In certain embodiments, the tissue comprises heart tissue. In certain instances, the tissue comprises lung tissue. In certain cases, the tissue comprises kidney tissue. In some embodiments, the tissue comprises ocular tissue. In some embodiments, the ocular tissue is corneal tissue. Organs of interest include those organs where blocking of Ang-2 protein is of interest. In some embodiments, the organ is a heart, a lung or a kidney.

As used herein, the term “tissue” refers to both transplanted tissue and graft bed tissue or any other tissue of a subject. In some instances, the tissue is a tissue that is transplanted in a subject (i.e., transplanted tissue), where the tissue may be foreign tissue or the subject's own tissue, e.g., heterologous, homologous, or isologous graft tissue. In other instances, the tissue is graft bed tissue of the subject. As used herein, the term “graft bed” refers to the site to which a graft or transplanted tissue is joined. In some embodiments of the methods, tissue includes transplanted tissue and/or associated secondary lymphoid tissue.

In some embodiments, the methods further include transplanting tissue in a graft bed of the subject. In some embodiments, the methods further include transplanting an organ into the subject. Any convenient methods of transplantation, including methods of preparing a graft bed and methods of transplanting an organ or tissue into the graft bed, may be adapted for use in the subject methods. The subject methods cam provide for reduction of inflammatory lymphangiogenesis at or around the grafting border between the transplanted tissue and the graft bed, e.g., in the tissue at or immediately adjacent to the grafting border.

Administration of the subject compositions (e.g., as described herein) may be performed at any convenient time before, during and/or after a transplantation procedure. In some cases, the administration is performed prior to transplantation. In some instances, the administration is performed during the transplantation procedure. In some embodiments, the administration is performed after the transplantation procedure.

In some cases the subject method, further comprises administering to the subject at least one additional compound. Any convenient agents may be utilized, including compounds useful for treating viral infections. The terms “agent,” “compound,” and “drug” are used interchangeably herein. In certain cases, the subject antagonist is administered to the subject in combination with an additional agent (e.g., an active agent that finds use in treatment of transplant rejection, or modulation of lymphangiogenesis). The antagonist and additional agent may be administered sequentially, simultaneously, or a combination thereof, as desired. As such, the antagonist and additional agent are administered in combination. In some instances, the antagonist and additional agent are administered simultaneously (together, at the same time) as a single composition or as separate compositions but at the same time. In some cases, the antagonist and additional agent are administered sequentially (e.g., administer one composition immediately after the other; or administer one composition and after a time period administer the other composition), as desired.

Antagonist to any convenient target molecules can be utilized in a combination therapy with the subject Ang-2 antagonists. In certain embodiments, the at least one additional compound is selected from an agent that specifically binds to tyrosine kinase receptor, such as a VEGFR, e.g., VEGFR-3, an agent that specifically binds to an integrin receptor, such as a VLA, e.g., VLA-1, a VEGFR-3 antagonist, a VLA-1 antagonist. In some instances, the at least one additional compound is selected from an antibody, nucleic acid that modulates expression of the target, e.g., a siRNA, a peptide, a scaffolded protein, a small molecule.

In some cases, the at least one additional compound is selected from an immunosuppressive agent and an agent that reduces lymphangiogenesis. In certain instances, the additional compound is and immunosuppressive agent selected from a glucocorticoid, a cytostatic (e.g., methotrexate, fluorouracil, azathioprine, and the like), an antibody (e.g., Atgam or thymoglobuline), an immunophilin acting drug (e.g., ciclosporin, tacrolimus, sirolimus), an interferon (e.g., IFN-beta), an opioid, a TNF-alpha binding protein (e.g., infliximab, etanercept or adalimumab), a mycophenolate, and finagolimod. In certain instances, the additional compound is an agent that reduces lymphangiogenesis and is selected from a VLA-1 antagonist (e.g., an anti-VLA-1 antibody or siRNA), a VEGFR-3 antagonist (e.g., an anti-VEGFR-3 antibody or siRNA) and a VEGF antagonist (e.g., an anti-VEGF antibody or siRNA).

In some embodiments, the at least one additional compound is an antibody. The antibody may be a humanized antibody. Any convenient antibodies that may be useful in the treatment of lymphangiogenesis, or immune suppression (e.g., to prevent transplant rejection), may be utilized in the subject methods in combination with the subject compounds. As such, some instances of the method include concomitant administration of an Ang-2 antagonist and an antibody.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the drug and composition at such time that both the known drug and the composition of the present invention will have a therapeutic effect. Such concomitant administration may involve concurrent (i.e. at the same time), prior, or subsequent administration of the drug with respect to the administration of a compound of the invention. A person of ordinary skill in the art would have no difficulty determining the appropriate timing, sequence and dosages of administration for particular drugs and compositions of the present invention.

In some embodiments, the compounds (e.g., Ang-2 antagonist and the at least one additional compound) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Aspects of the subject methods further include assessing modulation of lymphangiogenesis in the sample or subject to determine whether the Ang-2 modulator (e.g., antagonist) reduces lymphangiogenesis. The term “assessing” refers to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent. A determining step in the subject methods can be carried out by any one or more of a variety a protocols for characterizing the presence and extent of lymphangiogenesis, LEC inhibition, LEC tube formation inhibition, and/or inhibition of antigen presenting cell trafficking to draining lymph nodes. In some instances, the method includes a step of determining lymphangiogenesis in a sample using a lymphatic endothelial molecular marker, e.g., a detectable marker selected from vascular endothelial growth factor receptor-3 (VEGFR-3), lymphatic vessel endothelial hyaluronan receptor-1 (LYVE-1) and Prox-1. The assessing step can be performed before and/or after the contacting or administration of the subject Ang-2 modulator (e.g., antagonist).

Aspect of the present disclosure include a method for treating or preventing LG in a subject comprising, contacting the subject with an siRNA molecule antagonist of Ang-2 (e.g., as described herein) via local administration to relevant tissues or cells, for example, by administration of vectors or expression cassettes of the disclosure that provide siRNA molecules to relevant cells.

Methods of delivery of viral vectors include, but are not limited to, intravenous administration and administration directly into a patient's eye. Generally, AAV virions may be introduced into cells using either in vivo or in vitro transduction techniques. If transduced in vitro, the desired recipient cell will be removed from the subject, transduced with AAV virions and reintroduced into the subject. Alternatively, syngeneic or xenogeneic cells can be used where those cells will not generate an inappropriate immune response in the subject.

In some embodiments, pharmaceutical compositions will comprise sufficient genetic material to produce a therapeutically effective amount of the siRNA of interest, i.e., an amount sufficient to reduce or ameliorate symptoms of the disease state in question or an amount sufficient to confer the desired benefit. The pharmaceutical compositions may also contain a pharmaceutically acceptable excipient.

As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene could be expressed by the delivered viral vector. Alternatively, separate vectors, each expressing one or more different transgenes, can also be delivered as described herein. Furthermore, it is also intended that the viral vectors delivered by the methods of the present disclosure be combined with other suitable compositions and therapies.

The present disclosure further provides a nucleic acid (e.g., siRNA), an expression cassette and/or a vector as described herein for use in medical treatment or diagnosis, e.g., for treating LG.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes. The term “endogenous gene” refers to a native gene in its natural location in the genome of an organism.

“Wild-type” refers to the normal gene or organism found in nature.

A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest, for example an siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted herein, the term “suitable regulatory sequences” is not limited to promoters. However, some suitable regulatory sequences useful in the present disclosure will include, but are not limited to constitutive promoters, tissue-specific promoters, development-specific promoters, regulatable promoters and viral promoters.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present disclosure include the mouse U6 RNA promoters, synthetic human H1RNA promoters, SV40, CMV, RSV, RNA polymerase II and RNA polymerase III promoters.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild-type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the siRNA, or the like. Such elements may be included in the DNA as desired to obtain the optimal performance of the siRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein.

The recombinant DNA can be readily introduced into the host cells, e.g., mammalian, bacterial, yeast or insect cells by transfection with an expression vector composed of DNA encoding the siRNA by any procedure useful for the introduction into a particular cell, e.g., physical or biological methods, to yield a cell having the recombinant DNA stably integrated into its genome or existing as a episomal element, so that the DNA molecules, or sequences of the present disclosure are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed herein, a “transfected” “or “transduced” host cell or cell line is one in which the genome has been altered or augmented by the presence of at least one heterologous or recombinant nucleic acid sequence. The host cells of the present disclosure are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the siRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

The present disclosure provides a cell expression system for expressing exogenous nucleic acid material in a mammalian recipient. The expression system, also referred to as a “genetically modified cell,” comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into an anti-sense RNA, a siRNA.

The condition amenable to gene inhibition therapy may be a prophylactic process, i.e., a process for preventing disease or an undesired medical condition. Thus, the instant disclosure embraces a system for delivering siRNA that has a prophylactic function (i.e., a prophylactic agent) to the mammalian recipient.

Methods for Introducing the Expression Cassettes into Cells

The inhibitory nucleic acid material (e.g., an expression cassette encoding siRNA directed to a gene of interest) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the siRNA together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of siRNA generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding a siRNA sequence that are in the cell.

In addition to at least one promoter and at least one heterologous nucleic acid sequence encoding the siRNA, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the siRNA(s), the other vector containing a selection gene. The selection of a suitable promoter, enhancer, selection gene, and/or signal sequence is deemed to be within the scope of one of ordinary skill in the art without undue experimentation.

The instant disclosure provides methods for genetically modifying cells of a mammalian recipient in vivo. According to one embodiment, the method comprises introducing an expression vector for expressing an siRNA sequence in cells of the mammalian recipient in situ by, for example, injecting the vector into the recipient.

Delivery Vehicles for the Expression Cassettes

Delivery of compounds into tissues can be limited by the size and biochemical properties of the compounds. Currently, efficient delivery of compounds into cells in vivo can be achieved only when the molecules are small (usually less than 600 Daltons).

The selection and optimization of a particular expression vector for expressing a specific siRNA in a cell can be accomplished by obtaining the nucleic acid sequence of the siRNA, possibly with one or more appropriate control regions (e.g., promoter, insertion sequence); preparing a vector construct comprising the vector into which is inserted the nucleic acid sequence encoding the siRNA; transfecting or transducing cultured cells in vitro with the vector construct; and determining whether the siRNA is present in the cultured cells.

Vectors for cell gene therapy include viruses, such as replication-deficient viruses. Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present disclosure. Such retroviruses further have utility for the efficient transduction of nucleic acid sequences into cells in vivo. Retroviruses have been used extensively for transferring nucleic acid material into cells. Protocols for producing replication-deficient retroviruses (including the steps of incorporation of exogenous nucleic acid material into a plasmid, transfection of a packaging cell line with plasmid, production of recombinant retroviruses by the packaging cell line, collection of viral particles from tissue culture media, and infection of the target cells with the viral particles) are well known in the art.

An advantage of using retroviruses for gene therapy is that the viruses insert the nucleic acid sequence encoding the siRNA into the host cell genome, thereby permitting the nucleic acid sequence encoding the siRNA to be passed on to the progeny of the cell when it divides. Promoter sequences in the LTR region have can enhance expression of an inserted coding sequence in a variety of cell types. Some disadvantages of using a retrovirus expression vector are (1) insertional mutagenesis, i.e., the insertion of the nucleic acid sequence encoding the siRNA into an undesirable position in the target cell genome which, for example, leads to unregulated cell growth and (2) the need for target cell proliferation in order for the nucleic acid sequence encoding the siRNA carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells.

Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination results. The desired recombinants are identified by screening individual plaques generated in a lawn of complementation cells.

Application of siRNA is accomplished by transfection of synthetic siRNAs, in vitro synthesized RNAs, or plasmids expressing siRNAs. More recently, viruses have been employed for in vitro studies and to generate transgenic mouse knock-downs of targeted genes. Recombinant adenovirus, adeno-associated virus (AAV) and feline immunodeficiency virus (FIV) can be used to deliver genes in vitro and in vivo. Each has its own advantages and disadvantages. Adenoviruses are double stranded DNA viruses with large genomes (36 kb) and have been engineered to accommodate expression cassettes in distinct regions.

Adeno-associated viruses have encapsidated genomes, similar to Ad, but are smaller in size and packaging capacity (˜30 nm vs. ˜100 nm; packaging limit of ˜4.5 kb). AAV contain single stranded DNA genomes of the + or the −strand. Eight serotypes of AAV (1-8) have been studied extensively. An important consideration for the present application is that AAV5 transduces striatal and cortical neurons, and is not associated with any known pathologies.

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication. Physically, the parvovirus virion is non-enveloped and its icosohedral capsid is approximately 20 nm in diameter.

Further provided by this disclosure are chimeric viruses where AAV can be combined with herpes virus, herpes virus amplicons, baculovirus or other viruses to achieve a desired tropism associated with another virus. For example, the AAV4 ITRs could be inserted in the herpes virus and cells could be infected. Post-infection, the ITRs of AAV4 could be acted on by AAV4 rep provided in the system or in a separate vehicle to rescue AAV4 from the genome. Therefore, the cellular tropism of the herpes simplex virus can be combined with AAV4 rep mediated targeted integration. Other viruses that could be utilized to construct chimeric viruses include lentivirus, retrovirus, pseudotyped retroviral vectors, and adenoviral vectors.

Also provided by this disclosure are variant AAV vectors. For example, the sequence of a native AAV, can be modified at individual nucleotides. The present disclosure includes native and mutant AAV vectors. The present disclosure further includes all AAV serotypes.

FIV is an enveloped virus with a strong safety profile in humans; individuals bitten or scratched by FIV-infected cats do not seroconvert and have not been reported to show any signs of disease. Like AAV, FIV provides lasting transgene expression in mouse and nonhuman primate neurons, and transduction can be directed to different cell types by pseudotyping, the process of exchanging the virus's native envelope for an envelope from another virus.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (Promega®, Madison, Wis.). However, the efficiency of transfection by these methods is highly dependent on the nature of the target cell and accordingly, the conditions for optimal transfection of nucleic acids into cells using the herein-mentioned procedures must be optimized. Such optimization is within the scope of one of ordinary skill in the art without the need for undue experimentation.

### Utility

The methods, compositions and kits of the present disclosure, e.g., as described above, find use in a variety of applications. Applications of interest include, but are not limited to: research applications and therapeutic applications. Methods of the disclosure find use in a variety of different applications including any convenient application where modulation of the occurrence of lymphangiogenesis can be achieved by blocking the biological action of angiopoietin-2 (Ang-2). Applications of interest include, but are not limited to, any convenient applications where reduction of corneal inflammatory lymphangiogenesis (LG) is of interest, applications where inhibition of lymphatic endothelial cell (LEC) functions are of interest, and application where suppression of antigen presenting cell trafficking and/or immune response are of interest.

Lymphatic and immune system dysfunction has been found in myriad disorders from cancer metastasis to transplant rejection. The subject methods and compositions find use in a variety of therapeutic applications. Therapeutic applications of interest include, but are not limited to, treatment of transplant rejection (e.g., corneal transplant rejection), the treatment of lymphatic and/or immune related disease conditions, including neoplastic disease conditions (e.g., cancers), immune or autoimmune diseases, inflammatory diseases, traumas, chemical burns, infections, and tissue. There are many other disorders that are associated with a dysregulation of lymphatic and/or immune abnormalities.

The subject methods may be employed in the treatment of a variety of conditions where there is lymphangiogenesis and/or abherrant immune response. In certain embodiments, the lymphangiogenesis is inflammatory lymphangiogenesis. In such applications, an effective amount of the modulator is administered to the subject in need thereof. Treatment is used broadly, to include at least amelioration in one or more of the symptoms of the disease, as well as a complete cessation thereof, as well as a reversal and/or complete removal of the disease condition, i.e., a cure.

In some embodiments, the subject methods are employed in the treatment of subjects in need of tissue transplantation (e.g., heart, lung, kidney, ocular (e.g., corneal), etc.). In such applications, an effective amount of the Ang-2 antagonist is administered to the subject at any convenient time, before, during or after transplantation of the tissue. In such applications, the subject methods can suppress immune reaction and enhance survival of transplanted tissue in the subject.

The subject methods and compositions find use in a variety of transplantation applications, e.g., treatments to restore the functions of a tissue or an organ to patients whose other treatments have failed or who are experiencing medical emergencies. In some instances, the methods find use in treating or preventing immune-mediated rejection, which can reduce the risk of transplant failure. A variety of solid organ or tissue transplantation applications are envisaged, e.g., transplantation of tissues or organs where blood and lymphatic vessels are located, such as heart, kidney, lung, skin, eye, and the like.

In some embodiments, the methods and compositions find use in corneal transplantations. In certain embodiments, the corneal transplant patients have un-inflamed and avascular graft beds, which in some cases. can be characterized as low-risk of rejection. In certain instances, the corneal transplant patients have inflamed and highly vascularized corneas, which in some cases, can be characterized as having a high-risk of rejection, or as immune-compromised. In some cases, the subject methods prevent or treat lymphatic formation in the cornea after an inflammatory, traumatic, infectious, chemical, or toxic damage.

The subject methods find use in a variety of research applications. For example, the subject methods may be used to elucidate a synergistic relationship between the biological activities of two or more endogenous proteins of interest, e.g., synergistic relationships. The subject methods, compositions and kits may be used to screen for small molecule antagonists of Ang-2.

### Formulations, Dosage and Administration

Ang-2 antagonists (e.g., as described herein) find use in pharmaceutical compositions for modulating the occurrence of lymphangiogenesis in a subject, including methods of enhancing survival of transplanted tissue in a subject.

In some instances, the method further includes transplanting an organ into the subject. In some embodiments, the method further includes transplanting tissue in a graft bed of the subject. In some embodiments, the graft bed is inflamed and vascularized (e.g., high risk of rejection) when the tissue is transplanted to the graft bed. In some embodiments, the graft bed is un-inflamed and not vascularized (e.g., high risk of rejection) when the tissue is transplanted to the graft bed. Administering the Ang-2 antagonist can enhance survival of transplanted tissue in the subject.

In some cases, such treatment is achieved by administering to the subject an effective amount of the antagonist. For in vivo protocols, any convenient administration protocol may be employed. Depending upon the binding affinity of the antagonist, the response desired, the manner and site of administration, the half-life, the number of cells present, the type of target tissue, various protocols may be employed. The pharmaceutical compositions may be administered via any convenient method, such as but not limited to, parenterally, topically (e.g., by eye drops or transdermal patch), orally, subconjunctival, intraocular, periocular or retrobulbar administration. The pharmaceutical composition may be administered locally, e.g., to the graft bed of a transplant subject. The number of administrations will depend upon the factors described above. The pharmaceutical composition may be taken orally as a pill, powder, or dispersion; bucally; sublingually; injected intravascularly, intraperitoneally, intracranially subcutaneously; by inhalation, or the like. The precise dose and particular method of administration will vary and may be readily determined by the attending physician or human or animal healthcare provider, e.g., the dose and method may be determined empirically. The particular dosage of the antagonist for any application may be determined in accordance with the procedures used for therapeutic dosage monitoring, where maintenance of a particular therapeutic level is desired over an extended period of time, for example, greater than about two weeks, or where there is repetitive therapy, with individual or repeated doses of the antagonist over short periods of time, with extended intervals, for example, two weeks or more. A dose of the antagonist within a predetermined range would be given and monitored for response, so as to obtain a time-expression level relationship, as well as observing therapeutic response. Depending on the levels observed during the time period and the therapeutic response, one could provide a larger or smaller dose the next time, following the response. This process would be iteratively repeated until one obtained a dosage within the therapeutic range. Where the antagonist is chronically administered, once the maintenance dosage of the antagonist is determined, one could then do assays at extended intervals to be assured that the desired response was observed.

The antagonist can be formulated into preparations for injection by dissolving, suspending or emulsifying it in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The subject pharmaceutical excipients can include one or more pharmaceutically acceptable excipients. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations of the present disclosure can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, liquids, or foams, containing, in addition to the active ingredient, such carriers as are appropriate. In some embodiments, the topical formulation is an eye drop liquid formulation for administration to the eye. Any convenient components may be included in the eye drop formulation. In some embodiments, the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (i.e., sunblocking agents), etc.

Antagonists (e.g., as described herein) that find use in the subject methods may be formulated for topical administration. The vehicle for topical application may be in one of various forms, e.g. a lotion, cream, gel, ointment, stick, spray, liquid eye drops, or paste. They may contain various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant to disperse and suspend the discontinuous phase within the continuous phase.

Pharmaceutical compositions of interest may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers.

Unit dosage forms for injection or intravenous administration may include the antagonist in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of antagonist(s) calculated in an amount sufficient to produce the desired effect in association with a pharmaceutically acceptable diluent, carrier or vehicle. The specifications for the novel unit dosage forms of the present disclosure depend on the particular antagonist employed and the effect to be achieved, and the pharmacodynamics associated with each antagonist in the host.

Dose levels can vary as a function of the specific antagonist, the nature of the delivery vehicle, and the like. Desired dosages for a given antagonist are readily determinable by a variety of means. The dose administered to an animal, particularly a human, in the context of the present disclosure should be sufficient to effect a prophylactic or therapeutic response in the animal over a reasonable time frame, e.g., as described in greater detail below. Dosage will depend on a variety of factors including the strength of the particular antagonist employed, the condition of the animal, and the body weight of the animal, as well as the severity of the illness and the stage of the disease. The size of the dose will also be determined by the existence, nature, and extent of any adverse side-effects that might accompany the administration of a particular antagonist.

In pharmaceutical dosage forms, the antagonist may be administered in the form of a free base, their pharmaceutically acceptable salts, or they may also be used alone or in appropriate association, as well as in combination, with other pharmaceutically active compounds.

In some embodiments, the subject Ang-2 antagonists may be administered in combination with one or more additional compounds or therapies, including a second target-binding molecule, an immune suppressing agent, surgery, catheter devices, and radiation. Combination therapy includes administration of a single pharmaceutical dosage formulation which contains the subject antagonist and one or more additional agents; as well as administration of the subject antagonist and one or more additional agent(s) in its own separate pharmaceutical dosage formulation. For example, a subject antagonist and an additional agent (e.g., and agent active as an immune suppressant) can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

Aspects of the present disclosure include a pharmaceutical composition including an Ang-2 antagonist and at least one additional compound. The combination composition may include a pharmaceutically acceptable excipient as described herein. In some cases, the at least one additional compound is selected from an immunosuppressive agent and an agent that reduces lymphangiogenesis. In certain instances, the at least one additional compound is an immunosuppressive agent selected from a glucocorticoid, a cytostatic (e.g., methotrexate, fluorouracil, azathioprine, and the like), an antibody (e.g., Atgam or thymoglobuline), an immunophilin acting drug (e.g., ciclosporin, tacrolimus, sirolimus), an interferon (e.g., IFN-beta), an opiod, a TNF-alpha binding protein (e.g., infliximab, etanercept or adalimumab), a mycophenolate, and finagolimod. In certain instances, the at least one additional compound is an agent that reduces lymphangiogenesis and is selected from a VLA-1 antagonist (e.g., an anti-VLA-1 antibody or siRNA), a VEGFR-3 antagonist (e.g., an anti-VEGFR-3 antibody or siRNA) and a VEGF antagonist (e.g., an anti-VEGF antibody or siRNA).

### Kits

Aspects of the disclosure further include kits that find use in practicing the subject methods. In some embodiments, the kits for practicing the subject methods include one or more pharmaceutical formulations, which include one or more Ang-2 antagonists, e.g., as described herein. As such, in certain embodiments the kits may include a single pharmaceutical composition, present as one or more unit dosages.

Any of the components described herein may be provided in the kits. A variety of components suitable for use in practicing the subject methods may find use in the subject kits. Kits may also include one or more components including, but not limited to, a syringe suitable for intra-ocular injection, an eye numbing agent, a sterile dilution buffer and a sealed package configured to maintain the sterility of the ophthalmic pharmaceutical composition, sterile containers, pharmaceutically acceptable solutions, freeze-dried solids thereof, tubes, buffers, etc., and instructions for use. The various components of the kits may be present in separate containers, or some or all of them may be pre-combined into a mixture in a single container, as desired. In certain embodiments, the kit includes a sterile container containing a pharmaceutically acceptable solution comprising the Ang-2 modulator; and a sealed package configured to maintain the sterility of the sterile container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), hard drive etc., on which the information has been recorded. Yet another form of these instructions that may be present is a website address which may be used via the internet to access the information at a removed site. For example, a kit according to one embodiment includes as a first component (a) instructions for using a pharmaceutical composition, and as a second component (b) a pharmaceutical composition comprising the Ang-2 modulator.

### Example 1

### Role of Angiopoietin-2 in Corneal Lymphangiogenesis

Methods.

Standard suture placement model was used to study Ang-2 expression in inflamed cornea, and corneal inflammatory lymphangiogenesis (LG) and hemangiogenesis (HG) responses in Ang-2 knockout mice. Moreover, human skin lymphatic endothelial cell (LEC) culture system was used to examine the effect of Ang-2 gene knockdown on LEC functions using small interfering RNAs (siRNAs). The effect of siRNA treatment on corneal LG was also assessed in vivo.

Results.

Angiopoietin-2 was expressed on lymphatic vessels and macrophages in inflamed cornea. While corneal LG response was abolished in Ang-2 knockout mice, the HG response was also suppressed but to a lesser degree with disorganized patterning. Moreover, anti-Ang-2 treatment inhibited LEC proliferation and capillary tube formation in vitro and corneal LG in vivo.

### CONCLUSIONS

Angiopoietin-2 is critically involved in lymphatic processes in vivo and in vitro in different tissues and sites. Modulation of the Ang-2 pathway can provide therapeutic strategies for lymphatic-related disorders, which occur both inside and outside the eye.

### INTRODUCTION

Angiopoietin-2 (Ang-2) is a ligand belonging to the angiopoietin-Tie family. Its functions through the Tie-2 receptor are dependent on the cellular textures. (Harfouche R, Hussain S N. Signaling and regulation of endothelial cell survival by angiopoietin-2. Am J Physiol Heart Circ Physiol. 2006; 291: H1635-H1645; Kim I, Kim J H, Moon S O, Kwak H J, Kim N G, Koh G Y. Angiopoietin-2 at high concentration can enhance endothelial cell survival through the phosphatidylinositol 3′-kinase/Akt signal transduction pathway. Oncogene. 2000; 19: 4549-4552; Maisonpierre P C, Suri C, Jones P F, et al. Angiopoietin-2, a natural antagonist for Tie2 that disrupts in vivo angiogenesis. Science. 1997; 277: 55-60; Teichert-Kuliszewska K, Maisonpierre P C, Jones N, et al. Biological action of angiopoietin-2 in a fibrin matrix model of angiogenesis is associated with activation of Tie2. Cardiovasc Res. 2001; 49: 659-670; Yuan H T, Khankin E V, Karumanchi S A, Parikh S M. Angiopoietin 2 is a partial agonist/antagonist of Tie2 signaling in the endothelium. Mol Cell Biol. 2009; 29: 2011-2022). Ang-2 is expressed during lymphatic development at embryonic and neonatal stages, and Ang-2 knockout (KO) mice demonstrate severe defects in lymphatic patterning and clinical signs of chylous ascites and lymphedema. (Shimoda H. Immunohistochemical demonstration of Angiopoietin-2 in lymphatic vascular development. Histochem Cell Biol. 2009; 131: 231-238; Dellinger M, Hunter R, Bernas M, et al. Defective remodeling and maturation of the lymphatic vasculature in Angiopoietin-2 deficient mice. Dev Biol. 2008; 319: 309-320). However, the specific roles of Ang-2 in pathologic lymphatic processes still remain largely unknown. In this study, using both in vivo corneal inflammatory LG model, and in vitro human skin lymphatic endothelial cell (LEC) culture system, it is shown that: (1) Ang-2 is expressed on both lymphatic vessels and macrophages in inflamed cornea; (2) corneal LG response is almost abolished in inflamed cornea of Ang-2 gene knockout mice while the hemangiogenesis (HG) response is suppressed to a lesser degree with disorganized blood vessels; (3) Ang-2 gene knockdown by siRNAs markedly inhibits LEC functions of proliferation and tube formation in vitro; (4) anti-Ang-2 treatment by siRNAs is effective in suppressing corneal LG in vivo. These data together indicate that Ang-2 is critically involved in LG processes, and its manipulation offers a new therapeutic strategy to interfere with LG and related diseases.

### Methods

Animals

Mice were treated according to the ARVO Statement for the Use of Animals in Ophthalmic and Vision Research, and the protocols were approved by the Animal Care and Use Committee of the institutes. Mice were anesthetized using a mixture of ketamine, xylazine, and acepromazine (50 mg, 10 mg, and 1 mg/kg body weight, respectively) for each surgical procedure. Six- to eight-week-old male C57BL/6 mice (Taconic Farms, Germantown, N.Y., USA) were used for corneal RNA extraction and immunofluorescent microscopic assays. Angiopoietin-2 knockout (denoted as Ang-2−/−/KO; Regeneron Pharmaceuticals, Inc., Tarrytown, N.Y., USA) and control littermates (denoted as Ang-2+/+/wild-type [WT]) in the C57BL/6 background were generated and maintained, as described previously. (Dellinger M, Hunter R, Bernas M, et al. Defective remodeling and maturation of the lymphatic vasculature in Angiopoietin-2 deficient mice. Dev Biol. 2008; 319: 309-320).

Lymphatic Endothelial Cells and Antibodies

Human microdermal LECs were purchased from Lonza (Walkersville, Md., USA) and maintained in EGM-2 MV medium (Lonza) according to manufacturer's instructions. (Grimaldo S, Yuen D, Ecoiffier T, Chen L. Very late antigen-1 mediates corneal lymphangiogenesis. Invest Ophthalmol Vis Sci. 2011; 52: 4808-4812). The following primary and secondary antibodies and isotype controls were used: FITC-conjugated rat-anti-mouse CD31 antibody, goat IgG control, purified goat-anti-mouse or human Ang-2 antibody (Santa Cruz Biotechnology, Dallas, Tex., USA), purified rabbit-anti-mouse LYVE-1 antibody, purified rat-anti-mouse monoclonal F4/80 antibody (Abcam, Inc. Cambridge, Md., USA), purified rat-anti-mouse antibody (CD16/CD32 Fc Block; BD Biosciences, San Jose, Calif., USA), Cy3-conjugated donkey-anti-rabbit secondary antibody, Cy3-conjugated donkey-anti-goat secondary antibody, FITC-conjugated donkey-anti-goat secondary antibody, 488-conjugated donkey-anti-rat secondary antibody, 488-conjugated goat-anti-rabbit secondary antibody, normal goat serum, and normal donkey serum (DyLight; Jackson ImmunoResearch Laboratories, Inc. West Grove, Pa., USA).

Corneal Suture Placement

The standard suture placement model was used to induce corneal inflammatory LG and HG (Grimaldo S, Yuen D, Ecoiffier T, Chen L. Very late antigen-1 mediates corneal lymphangiogenesis. Invest Ophthalmol Vis Sci. 2011; 52: 4808-4812; Yuen D, Pytowski B, Chen L. Combined blockade of VEGFR-2 and VEGFR-3 inhibits inflammatory lymphangiogenesis in early and middle stages. Invest Ophthalmol Vis Sci. 2011; 52: 2593-2597). Briefly, three interrupted sutures (11-0 nylon, Arosurgical, Newport Beach, Calif., USA) were placed into corneal stroma without penetrating into the anterior chamber. Sutures were left in place for 2 weeks before the corneas were sampled for further analysis. Experiments were repeated twice with six mice in each group.

Therapeutic Intervention with Ang-2 siRNA

Six- to eight-week-old male C57BL/6 mice after suture placement were randomly selected to receive subconjunctival injection of 5 μL (0.2 μg/μL) Ang-2 specific siRNA (Invitrogen, Carlsbad, Calif., USA) or control twice a week for 2 weeks when whole-mount corneas were harvested for immunofluorescent microscopic analysis. Experiments were repeated twice with six mice in each group.

Corneal Immunofluorescent Microscopy and Slit-Lamp Biomicroscopy

The experiments were performed according to our standard protocol. (Chen et al., Very late antigen-1 mediates corneal lymphangiogenesis. Invest Ophthalmol Vis Sci. 2011; 52: 4808-4812; Chen et al., Combined blockade of VEGFR-2 and VEGFR-3 inhibits inflammatory lymphangiogenesis in early and middle stages. Invest Ophthalmol Vis Sci. 2011; 52: 2593-2597; Chen et al., Increased lymphangiogenesis and hemangiogenesis in infant cornea. Lymphat Res Biol. 2011; 9: 109-114). Briefly, freshly excised wholemount corneas at day 14 after suture placement were fixed in acetone for immunofluorescent staining against LYVE-1, CD31, F4/80. For Ang-2 staining, the samples were stained with purified goat-anti-mouse Ang-2 antibodies, visualized by the Cy3-conjugated donkey-anti-goat or FITC-conjugated donkey-anti-goat secondary antibodies. Samples were covered with mounting medium (Vectashield; Vector Laboratories, Burlingame, Calif., USA) and digital images were taken by an epifluorescence microscope with software (AxioImager M1 with AxioVision 4.8; Carl Zeiss A G, Gottingen, Germany). Additionally, corneal blood vessels were examined by an ophthalmic slit-lamp with an integrated digital camera system (SL-D4 and DC-3; Topcon Medical Systems, Tokyo, Japan).

Vascular Quantification

Corneal blood and lymphatic vessels were analyzed using ImageJ software (http://imagej.nih.gov/W; provided in the public domain by the National Institutes of Health, Bethesda, Md., USA). (Chen et al., Very late antigen-1 mediates corneal lymphangiogenesis. Invest Ophthalmol Vis Sci. 2011; 52: 4808-4812; Chen et al., Increased lymphangiogenesis and hemangiogenesis in infant cornea. Lymphat Res Biol. 2011; 9: 109-114; Dietrich T, Onderka J, Bock F, et al. Inhibition of inflammatory lymphangiogenesis by integrin alpha5 blockade. Am J Pathol. 2007; 171: 361-372). Vascular structures stained as CD31+LYVE-1− were identified as blood vessels, whereas those stained as CD31+LYVE-1+ were defined as lymphatic vessels. The percentage scores of LG coverage areas were obtained by normalizing to control groups where the lymphatic coverage areas were defined as being 100%. The blood vessels were evaluated using three parameters: invasion distance, branching points, and vessel caliber. (Chen et al., Increased lymphangiogenesis and hemangiogenesis in infant cornea. Lymphat Res Biol. 2011; 9: 109-114) The percentage scores of the invasion distance were obtained by normalizing to the distance from the limbus to the suture site in the control groups which was defined as being 100%. Diameters and branching points were quantified by averaging five randomly chosen areas in the samples, as reported previously. (Chen et al., Increased lymphangiogenesis and hemangiogenesis in infant cornea. Lymphat Res Biol. 2011; 9: 109-114).

Corneal Macrophage Isolation

Corneal macrophages were isolated. (Chen et al., Very late antigen-1 mediates corneal lymphangiogenesis. Invest Ophthalmol Vis Sci. 2011; 52: 4808-4812; Hamrah P, Liu Y, Zhang Q, Dana M R. The corneal stroma is endowed with a significant number of resident dendritic cells. Invest Ophthalmol Vis Sci. 2003; 44: 581-589) Briefly, 20 corneal buttons from normal or sutured corneas were harvested and cultured in RPMI-1640 with 10% FBS, 10 mmol/L HEPES, 0.1 mM nonessential amino acid, 1×10-5 mol/L 2-mercaptoethanol, 1 mmol/L sodium pyruvate, 100 U/mL penicillin, and 100 μg/mL streptomycin (GIBCO; Invitrogen) at 37° C. for 1 week. Nonadherent cells were discarded and adherent cells of macrophage population were collected for RNA extraction.

Real-time Quantitative Reverse Transcription PCR

The assays were performed to measure the expression levels of Ang-2 and GAPDH, as reported previously. (Chen et al., Very late antigen-1 mediates corneal lymphangiogenesis. Invest Ophthalmol Vis Sci. 2011; 52: 4808-4812) Total RNA was extracted with Trizol (Invitrogen) from corneal macrophages and subjected to reverse transcription with the cDNA synthesis kit (iScript; Bio-Rad, Hercules, Calif., USA) according to the manufacturer's instructions. Quantitative analyses of expression levels were performed with the CFX96 real-time polymerase chain reaction (RT-PCR) machine (Bio-Rad). Expression levels of mature miRNAs were quantified with supermix (SsoFast EvaGreen Supermix; Bio-Rad) following manufacturer's instructions. An average of three experiments was performed in triplicate, with standard deviation presented. Primer sets were: GAPDH: 5′-AAGGTGAAGGTCGGAGTC-3′ (SEQ ID NO:3) and 5′-GATTTTGGAGGGATCTCG-3′ (SEQ ID NO:4); Ang-2: 5′-GCTTCGGGAGCCCTCTGGGA-3′ (SEQ ID NO:5) and 5′-TGAGCGAGTAGCCGGACCCC-3′ (SEQ ID NO:6).

Immunocytofluorescent Microscopic Assay

For LEC staining, the experiments were performed (Chen et al., Very late antigen-1 mediates corneal lymphangiogenesis. Invest Ophthalmol Vis Sci. 2011; 52: 4808-4812). Briefly, cells were seeded on culture slide chamber (BD Biosciences) and fixed in acetone for staining with purified goat-anti-mouse Ang-2 antibody, which was visualized by Cy3-conjugated donkey-anti-goat secondary antibody. Samples were mounted with mounting medium with DAPI (Vector Laboratories), and digital images were taken with an epifluorescence microscope (Zeiss Axioplan 2; Carl Zeiss Meditec).

Reverse Transcriptase PCR

The experiments were performed as described by Chen et al. (Chen et al., Very late antigen-1 mediates corneal lymphangiogenesis. Invest Ophthalmol Vis Sci. 2011; 52: 4808-4812; Chen et al., Novel characterization of lymphatic valve formation during corneal inflammation. PLoS One. 2011; 6: e21918). Total RNA was extracted and purified from LECs with an RNA kit (RNAeasy mini-kit; Qiagen, Valencia, Calif., USA). Reverse transcription was performed using the cDNA synthesis kit from Invitrogen (SuperScript VILO). Polymerase chain reaction was performed with the PCR mastermix from Promega (Madison, Wis., USA). All thermal cycles were carried out in a PCR system (Mastercycler ep; Eppendorf, Germany). Primer sequences were: Ang-2, forward 5′-GCTTCGGGAGCCCTCTGGGA-3′ (SEQ ID NO:7), reverse 5′-TGAGCGAGTAGCCGGACCCC-3′ (SEQ ID NO:8); GAPDH, forward 5′-CCACAGTCCATGCCATCAC-3′ (SEQ ID NO:9), reverse 5′-TCCACCACCCTGTTGCTGT-3′ (SEQ ID NO:10).

Ang-2 siRNA Transfection

The experiment was performed as reported by Chen et al. (Grimaldo S, Yuen D, Ecoiffier T, Chen L. Very late antigen-1 mediates corneal lymphangiogenesis. Invest Ophthalmol Vis Sci. 2011; 52: 4808-4812). Custom designed siRNA duplexes were synthesized by Applied Biosystems (Foster City, Calif., USA). The siRNA sequences were designed against human Ang-2 mRNA: sense 5′-CGUUAACAUUCCCUAAUUCtt-3′ (SEQ ID NO:1); antisense 5′-GAAUUAGGGAAUGUUAACGtg-3′ (SEQ ID NO:2). A scrambled siRNA control was purchased from Ambion (Austin, Tex., USA). Transfections were carried out according to manufacturer's instructions with a transfection reagent (RNAiMax; Invitrogen) and opti-MEM reduced serum medium at 37° C. in a 5% CO2 humidified air incubator.

Three-Dimensional Culture and Tube Formation Assay

The experiment was performed as described by Chen et al. (Grimaldo S, Yuen D, Ecoiffier T, Chen L. Very late antigen-1 mediates corneal lymphangiogenesis. Invest Ophthalmol Vis Sci. 2011; 52: 4808-4812). Forty-eight hours following siRNA transfection, LECs were reseeded (30,000 cells/well) onto 48-well plates containing solidified matrigel (BD Biosciences) and monitored for 24 hours under an inverted microscope (Zeiss Axio Observer A1; Carl Zeiss, Inc., Germany). Phase images of tubes were taken and total tube lengths were analyzed by imaging software (Qcapture; QImaging, British Columbia, Canada). Assays were performed in triplicate and repeated at least three times.

Proliferation Assay

LECs were seeded on collagen type I-coated 96-well plates (Chen et al., Very late antigen-1 mediates corneal lymphangiogenesis. Invest Ophthalmol Vis Sci. 2011; 52: 4808-4812). Forty-eight hours following siRNA transfection, the cells were subjected to a MTS proliferation assay from Promega (Madison, Wis., USA) according to the manufacturer's protocol. Assays were performed in duplicate and repeated at least three times.

Statistical Analysis

The results are reported as mean±SEM unless otherwise indicated, and Student t-test was used for the determination of significance levels between different groups using scientific graphing software (Prism; GraphPad, La Jolla, Calif., USA). The differences were considered statistically significant when P<0.05.

### Results

Ang-2 Expression in Inflamed Cornea

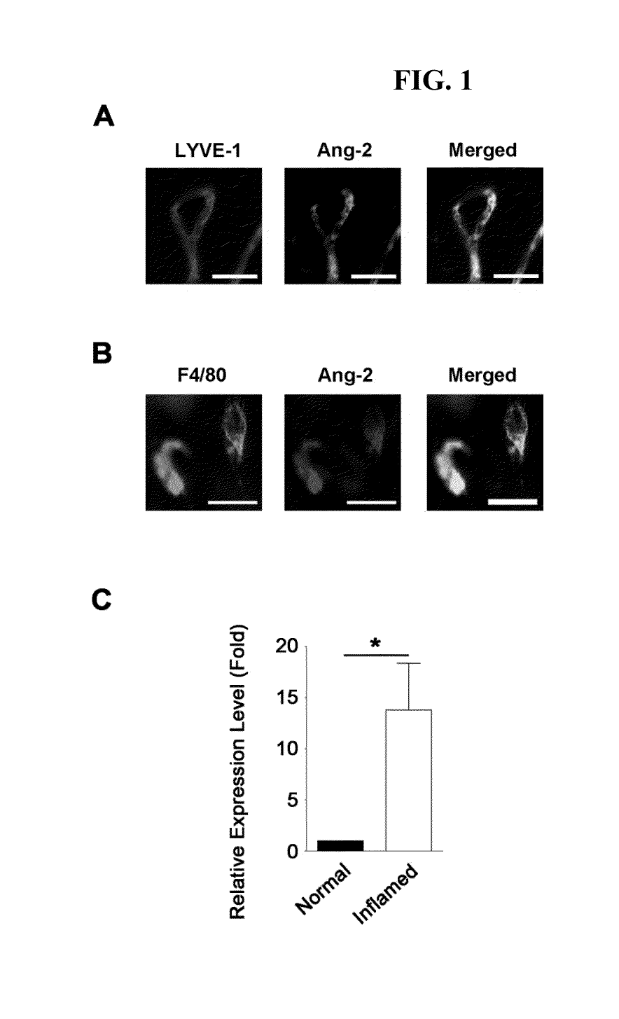

To study the role of Ang-2 in corneal inflammatory LG, Ang-2 expression was first assessed in inflamed mouse cornea using the suture placement model. As shown in FIG. 1, panel A by corneal whole-mount immunofluorescent microscopic assays, Ang-2 expression was confirmed on newly formed LYVE-1+ lymphatic vessels in the inflamed cornea. Moreover, Ang-2 was also detected on corneal macrophages, which coexpressed F4/80+(FIG. 1, panel B). Since it is known that macrophages are involved in LG in both corneal and noncorneal tissues, we next performed quantitative real time PCR (qPCR) assay and showed that Ang-2 expression on corneal macrophages was significantly elevated in inflamed condition (FIG. 1, panel C; *P<0.05).

Abolished Corneal LG Response in Ang-2 Knockout Mice

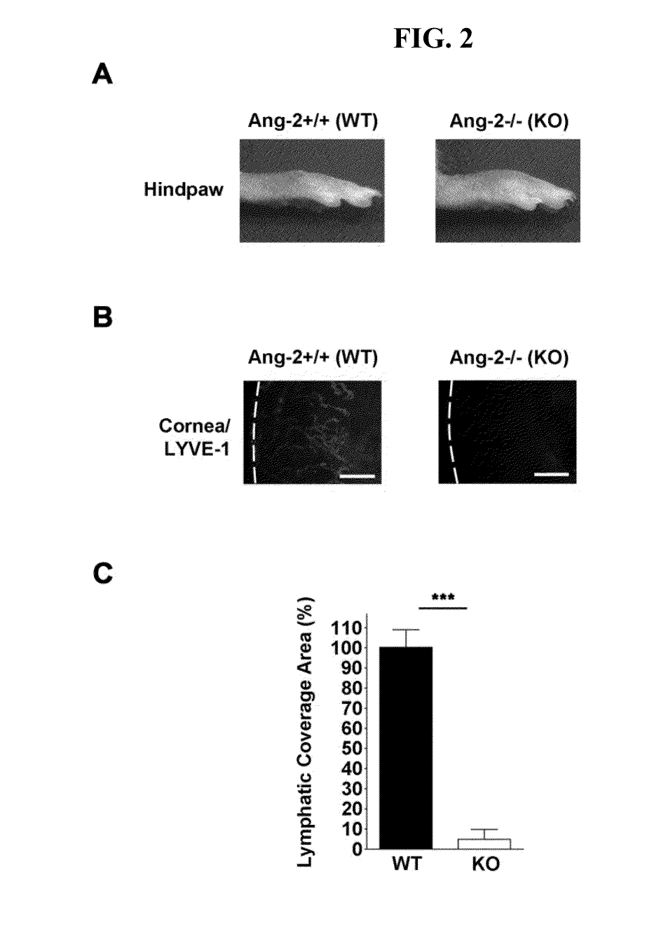

Corneal LG response in Ang-2 knockout (Ang-2−/−) mice was next examined using immunofluorescent microscopic assay and the suture placement model. As shown in FIG. 2, panel A, Ang-2 knockout mice demonstrated swollen hind paws reflecting lymphedema caused by inadequate lymphatic development. Results from our corneal assays showed that inflammatory LG response in Ang-2 knockout mice was nearly 100% inhibited compared with control littermates (FIG. 2, panel B). Summarized data on lymphatic coverage area from both knockout and control littermate mice are presented in FIG. 2, panel C (***P<0.001). These data indicate that Ang-2 gene depletion almost abolished LG responses in the inflamed cornea.

Suppressed Corneal HG Response in Ang-2 Knockout Mice with Disorganized Patterning

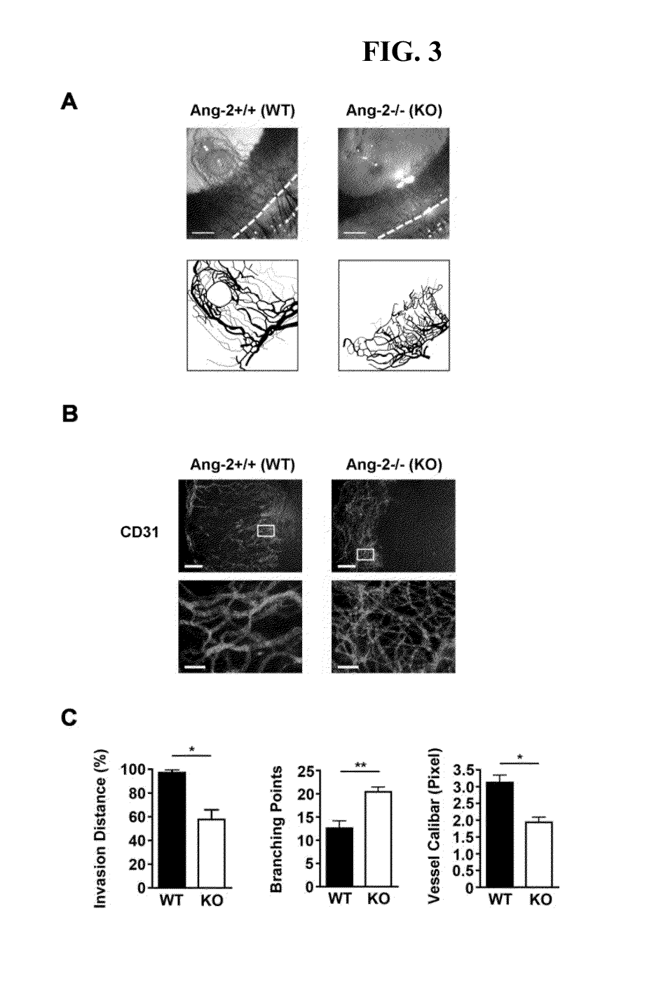

Aside from the absence of lymphatic vessels, significant suppression of HG response in inflamed corneas of Ang-2 knockout mice by both ophthalmic slit-lamp biomicroscopic examination (FIG. 3, panel A) and immunofluorescent microscopic assays (FIG. 3, panel B) was also observed. Moreover, the newly formed blood vessels in Ang-2 knockout mice demonstrated abnormal patterning with disorganized network, as shown in the enlarged boxed areas in FIG. 3B. Further analysis on these vessels revealed their shorter invasion distance into central cornea, more branching points, and smaller calibers as well, compared to control littermates (FIG. 3, panel C; *P<0.05; **P<0.01).

Downregulation of Ang-2 Expression in LECs by siRNA

To further investigate the specific role of Ang-2 in processes of LG, a human LEC culture system was employed, and Ang-2 expression was confirmed on these cells by both RT-PCR and immunocytofluorescent microscopic assays, as shown in FIG. 4, panel A and B. Whether Ang-2 expression in LECs can be downregulated by a siRNA-mediated gene silencing approach was next investigated. Lymphatic endothelial cells were transfected with either Ang-2 or scrambled siRNA. Forty-eight hours after the transfection, the depletion of Ang-2 expression in LECs was confirmed by RT-PCR analysis, as shown in FIG. 4, panel C. These results indicate that it is possible to use the siRNA approach to study functional roles of Ang-2 in LECs in vitro, as presented below.

Suppression of LEC Proliferation by Ang-2 Gene Knockdown

The effect of Ang-2 gene knockdown on LEC proliferation using the siRNA approach was next assessed. Forty-eight hours following siRNA transfection with either Ang-2 or scrambled siRNA, LECs were subjected to a MTS proliferation assay (Chen et al., Very late antigen-1 mediates corneal lymphangiogenesis. Invest Ophthalmol Vis Sci. 2011; 52: 4808-4812). As presented in FIG. 4, panel D, the results from this experiment showed that with Ang-2 gene knockdown in LECs, cell proliferation function was significantly reduced, compared with the control condition (***P<0.001).

Suppression of LEC Tube Formation by Ang-2 Gene Knockdown

The effect of Ang-2 siRNA treatment on LEC capillary tube formation in vitro was also investigated using the three-dimensional culture system (Chen et al., Very late antigen-1 mediates corneal lymphangiogenesis. Invest Ophthalmol Vis Sci. 2011; 52: 4808-4812). Forty-eight hours following the transfection with either Ang-2 or scrambled siRNA, LECs were seeded on matrigel to allow for capillary-type tube formation. The results showed that Ang-2 is critically involved in this important function of LECs as well. As demonstrated in FIG. 4, panel E and summarized from repetitive assays in FIG. 4, panel F, Ang-2 depletion led to a significant reduction in the total tubule length formed by the LECs (**P<0.01).

Suppression of Corneal Lymphangiogenesis by Ang-2 Inhibition

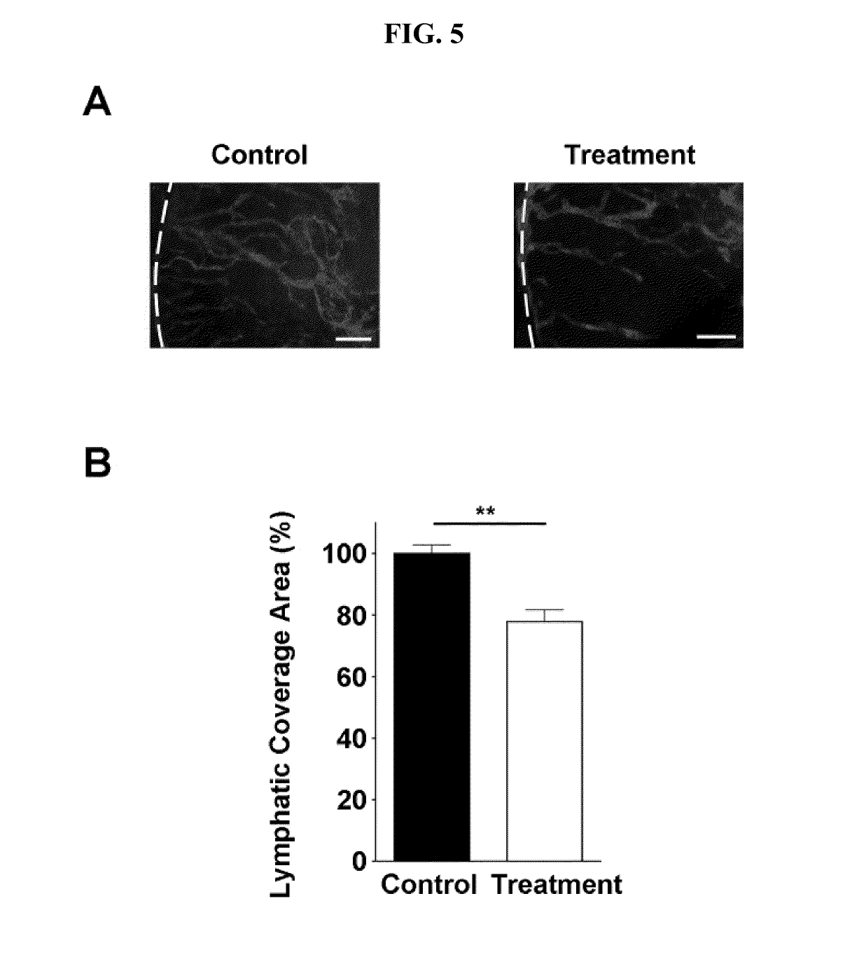

To further assess the in vivo effect of Ang-2 inhibition on corneal LG, the suture placement model was used and the effect of subconjunctival delivery of Ang-2 siRNA on corneal LG response was evaluated. As showed in FIG. 5, panel A, corneal LG was significantly suppressed in the anti-Ang-2 treatment group. Summarized data on lymphatic coverage area are presented in FIG. 5, panel B (**P<0.01). These data indicate that Ang-2 targeting is effective in suppressing corneal LG in vivo.

### DISCUSSION

In summary, this study shows that Ang-2 is critically involved in LG processes in vivo and in vitro. The fact that corneal LG response is almost eliminated in Ang-2 knockout mice indicates its role as a strong and determining factor of corneal LG response, which is supported by the anti-Ang-2 treatment data in wild-type mice.

This study suggests that the molecular mechanisms of corneal LG are more complex than previously considered. The study also reveals an interesting phenomenon that Ang-2 plays differential roles in corneal LG versus HG. While blood vessels are formed but disorganized in Ang-2 knockout mice, almost no lymphatic vessels are found in the same corneas. This indicates that LG is more susceptible than HG to Ang-2 gene depletion. Without Ang-2, lymphatic vessels are not even able to form. The stimulatory role of Ang-2 on lymphatic formation is also confirmed by the in vitro data showing that Ang-2 enhances LEC tube formation. It is also possible that Ang-2 plays an indispensible role in early stage LG response while it is more involved in the middle or late stages of HG when remodeling and maturation of blood vessels occur. In this study, no limbal lymphatics in inflamed Ang-2 knockout mice was observed, and it is yet to be determined whether a developmental defect exists in limbal lymphatics and how this affects pathologic LG in the knockout mice. The underlying mechanisms governing the disparity between LG and HG responses warrant further investigation as well.

Moreover, the modality by which Ang-2 triggers the prolymphangiogenic response in the cornea needs further exploration. A couple of developmental studies demonstrated the capacity of Ang-1 to compensate for the absence of Ang-2 in lymphatic remodeling in noncorneal tissues. (Dellinger M, Hunter R, Bernas M, et al. Defective remodeling and maturation of the lymphatic vasculature in Angiopoietin-2 deficient mice. Dev Biol. 2008; 319: 309-320; Gale N W, Thurston G, Hackett S F, et al. Angiopoietin-2 is required for postnatal angiogenesis and lymphatic patterning, and only the latter role is rescued by angiopoietin-1. Dev Cell. 2002; 3: 411-423). It was shown that both Ang-1 and Ang-2 are agonists in Tie-2 signaling and act positively in the process. These studies show how the genetic rescue of Ang-2 knockout, by substituting Ang-2 with Ang-1 sequence, is able to normalize the lymphatic vasculature. However, there is no demonstrated endogenous compensation of Ang-2 stimulation by higher Ang-1 expression within the tissue. While it is yet to be determined any roles of Ang-1 in corneal LG, based on our data with the Ang-2 knockout mice, Ang-1 seems not potent enough to compensate for the loss of Ang-2 via the endogenous pathway if it is present.

These results indicate the abnormal patterning of blood vessels in inflamed corneas of Ang-2 knockout mice. This observation on the pathological blood vessels is consistent with a showing that blood vessels formed during the development of Ang-2 knockout mice are defective in remodeling (Gale N W, Thurston G, Hackett S F, et al. Angiopoietin-2 is required for postnatal angiogenesis and lymphatic patterning, and only the latter role is rescued by angiopoietin-1. Dev Cell. 2002; 3: 411-423). Moreover, the finding that HG response is suppressed in Ang-2 knockout mice is consistent with reports that Ang-2 blockade inhibits HG response induced by FGF implantation in rat cornea or HG response associated with tumor growth (White R R, Shan S, Rusconi C P, et al. Inhibition of rat corneal angiogenesis by a nuclease-resistant RNA aptamer specific for angiopoietin-2. Proc Natl Acad Sci USA. 2003; 100: 5028-5033; Oliner J, Min H, Leal J, et al. Suppression of angiogenesis and tumor growth by selective inhibition of angiopoietin-2. Cancer Cell. 2004; 6: 507-516). It is also consistent with a showing that Ang-2 and VEGF pellet implantation induces significant HG response in the cornea. (Asahara T, Chen D, Takahashi T, et al. Tie2 receptor ligands, angiopoietin-1 and angiopoietin-2, modulate VEGF-induced postnatal neovascularization. Circ Res. 1998; 83: 233-240). However, these studies yielded no information on the aspect of lymphatic vessels.

The mechanisms of corneal LG are important since LG accompanies many corneal diseases after inflammatory, chemical, infectious, immunogenic, or traumatic damage, and LG is a major risk factor for corneal transplant rejection. Corneas enriched with LG are hostile to transplants for vision restoration due to a high rejection rate of 50%-90%, irrespective of current treatment modality. Unfortunately, many patients who are blind from corneal diseases fall into this high rejection category.

### Example 2

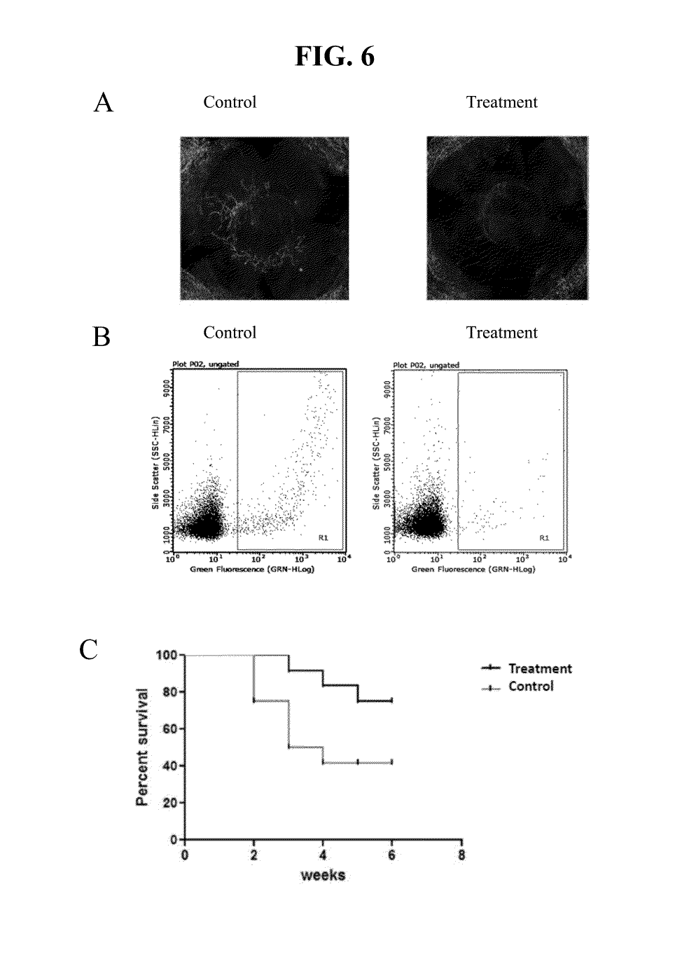

Anti-Ang-2 antibody treatment suppresses transplantation-induced corneal lymphangiogenesis and donor derived cell trafficking (i.e., antigen presenting cell trafficking) to draining lymph nodes after transplantation and promotes corneal graft survival (FIG. 6). Transplantation experiments were performed in normal mouse corneas (i.e., low risk of transplant rejection) and the in vivo effect of systemic administration of anti-Ang-2 antibody (Eli Lilly) was evaluated. Panel A: whole mount corneal images showing reduced lymphatic vessels (green) in recipient cornea in the treatment group. Panel B: flow cytometry analysis showing reduced antigen presenting cell trafficking to recipient draining lymph nodes in the treatment group. These results provide additional data (panel B) showing that anti-Ang-2 antibody treatment reduces or inhibits antigen presenting cell trafficking to recipient draining lymph nodes, an important aspect of immune response to foreign antigens. Panel C: Kaplan-Meier survival curves showing increased graft survival in the treatment group.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### Embodiments

Aspects of the present disclosure include a method of reducing the occurrence of lymphangiogenesis in a corneal transplant subject. In some embodiments, the method comprises administering to the subject an amount of an Ang-2 antagonist effective to reduce the occurrence of lymphangiogenesis in the subject. In some cases, the method is a method of improving graft survival in the subject. In some cases, the method is a method of reducing antigen presenting cell trafficking to draining lymph nodes. In certain embodiments, the subject has a high risk of corneal transplant rejection. In certain embodiments, the subject has an inflamed and vascularized graft bed. In certain embodiments, the subject has a low risk of corneal transplant rejection. In certain embodiments, the subject has an un-inflamed and avascular graft bed.

In some embodiments, the Ang-2 antagonist comprises a specific binding member that specifically binds to Ang-2. In certain embodiments, the Ang-2 antagonist is selected from a neutralizing antibody or binding fragment thereof, a scaffolded protein binder, a small molecule and a peptide. In some embodiments, the Ang-2 antagonist selectively reduces expression of Ang-2. In certain embodiments, the Ang-2 antagonist comprises a nucleic acid. In certain embodiments, the Ang-2 antagonist is a RNAi molecule. In some cases, the RNAi molecule comprises a nucleic acid having at least 90% sequence identity to SEQ ID NO:1.

In some embodiments of the method, the subject is a mammal. In certain embodiments the subject is human. In certain embodiments, the subject is a mouse. In certain embodiments, the administering comprises subconjunctival, intraocular, periocular, retrobulbar or topical administration. In some embodiments, the method further comprises transplanting corneal tissue in a graft bed of the subject. In certain embodiments, the administering is performed prior to the transplanting. In certain embodiments, the administering is performed during the transplanting. In certain embodiments, the administering is performed after the transplanting. In some embodiments, the method comprises reducing inflammatory lymphangiogenesis in the graft bed.

In certain embodiments, the method comprises preventing inflammatory lymphangiogenesis in the graft bed. In certain embodiments, the method comprises reducing inflammatory lymphangiogenesis around the grafting border between the transplanted tissue and the graft bed. In certain embodiments, the method comprises reducing inflammatory lymphangiogenesis at the grafting border between the transplanted tissue and the graft bed. In certain embodiments, the method comprises reducing the occurrence of opacity of the corneal tissue. In certain embodiments, the method does not significantly inhibit blood vessel growth in the tissue. In certain embodiments, the method comprises inhibiting lymphatic vessel growth in the tissue. In certain embodiments, the method comprises enhancing survival of the transplanted corneal tissue in the subject. In some embodiments, the method further includes assessing the subject for enhanced survival of the transplanted corneal tissue. In some embodiments, the method further includes assessing the subject for reduction of opacity of the corneal tissue. In some embodiments, the method further includes assessing the tissue for inhibition of lymphatic vessel growth. In some embodiments, the method further includes assessing the reduction of inflammatory lymphangiogenesis in the tissue.

Aspects of the present disclosure includes a method of inhibiting lymphatic endothelial cell (LEC) proliferation; and/or capillary tube formation; and/or inhibiting immune cell (e.g., antigen presenting cell) trafficking to draining lymph nodes, in a sample (e.g., a cellular sample or a tissue sample) comprising: contacting the sample with an effective amount of an Ang-2 antagonist to inhibit LEC proliferation. In certain embodiments, the sample is a tissue sample that comprises a graft bed. In certain embodiments, the tissue comprises transplanted tissue. In certain embodiments, the tissue comprises heart tissue, lung tissue, kidney tissue, skin tissue or ocular tissue. In certain embodiments, the tissue is corneal tissue. In certain embodiments, the tissue is inflamed and vascularized. In certain embodiments, the tissue is un-inflamed and avascular. In certain embodiments, the tissue is in vivo in a mammal. In certain embodiments, the contacting comprises administering a pharmaceutical composition comprising the Ang-2 antagonist to a mammal. In certain embodiments, the Ang-2 antagonist comprises a specific binding member that specifically binds to Ang-2. In certain embodiments, the Ang-2 antagonist is selected from a neutralizing antibody or binding fragment thereof, a scaffolded protein binder, a small molecule and a peptide. In certain embodiments, the Ang-2 antagonist selectively reduces expression of Ang-2. In certain embodiments, the Ang-2 antagonist comprises a nucleic acid. In certain embodiments, the Ang-2 antagonist is a RNAi molecule. In certain embodiments, the administration is via local administration. In certain embodiments, the administration is by subconjunctival, intraocular, periocular, retrobulbar, or topical administration. In certain embodiments, the method inhibits capillary tube formation. In certain embodiments, the method inhibits corneal lymphangiogenesis.

In some embodiments, the method further includes assessing the contacted sample for inhibition of lymphatic endothelial cell (LEC) proliferation, and/or capillary tube formation, and/or inhibiting antigen presenting cell trafficking to draining lymph nodes.

Aspects of the present disclosure include a pharmaceutical composition including an Ang-2 antagonist (e.g., as described herein) and an additional agent (e.g., as described herein). The composition may optionally include one or more pharmaceutically acceptable excipients (e.g., as described herein).

Aspects of the present disclosure include a kit comprising: an ophthalmic pharmaceutical composition comprising an antagonist of Ang-2; and one or more components selected from a syringe suitable for intra-ocular injection, an eye numbing agent, a sterile dilution buffer and a sealed package configured to maintain the sterility of the ophthalmic pharmaceutical composition.

What is claimed is:

1. A method of reducing the occurrence of lymphangiogenesis in a corneal transplant subject, the method comprising administering to the subject an amount of an Ang-2 antagonist effective to reduce the occurrence of lymphangiogenesis in the subject.

2. The method according to claim 1, wherein the subject has a high risk of corneal transplant rejection.

3. The method according to claim 2, wherein the subject has an inflamed and vascularized graft bed.

4. The method according to claim 1, wherein the subject has a low risk of corneal transplant rejection.

5. The method according to claim 4, wherein the subject has an un-inflamed and avascular graft bed

6. The method according to claim 1, wherein the Ang-2 antagonist comprises a specific binding member that specifically binds to Ang-2.

7. The method according to claim 6, wherein the Ang-2 antagonist is selected from a neutralizing antibody or binding fragment thereof, a scaffolded protein binder, a small molecule and a peptide.

8. The method according to claim 1, wherein the Ang-2 antagonist selectively reduces expression of Ang-2.

9. The method according to claim 8, wherein the Ang-2 antagonist is a RNAi molecule.

10. The method according to claim 1, wherein the subject is human.

11. The method according to claim 1, further comprising transplanting corneal tissue in a graft bed of the subject.

12. The method according to claim 11, wherein the administering is performed prior to the transplanting.

13. The method according to claim 11, wherein the administering is performed during the transplanting.

14. The method according to claim 11, wherein the administering is performed after the transplanting.

15. The method according to claim 11, wherein the method comprises reducing or preventing lymphangiogenesis in the graft bed.

16. The method according to claim 11, wherein the method comprises reducing lymphangiogenesis around the grafting border between the transplanted tissue and the graft bed.

17. The method according to claim 1, wherein the method does not significantly inhibit blood vessel growth in the tissue.

18. The method according to claim 1, wherein the method comprises suppressing antigen presenting cell trafficking to draining lymph nodes in immune reaction.

19. The method according to claim 11, wherein the method comprises enhancing survival of the transplanted corneal tissue in the subject.

20. A kit, comprising: an ophthalmic pharmaceutical composition comprising an antagonist of Ang-2; and one or more components selected from a syringe suitable for intra-ocular injection, an eye numbing agent, a sterile dilution buffer and a sealed package configured to maintain the sterility of the ophthalmic pharmaceutical composition.
